(19) United States
(12) Patent Application Publication
(10) Pub. No.: US 2021/0207089 A1
(43) Pub. Date: Jul. 8, 2021

(54) HEMORRHAGIC CEREBROSPINAL FLUID NEURAL STEM CELLS

(71) Applicants: SERVICIO ANDALUZ DE SALUD, Sevilla (ES); FUNDACIÓN PUBLICA ANDALUZA PROGRESO Y SALUD, Sevilla (ES); UNIVERSIDAD DE MÁLAGA, Málaga (ES)

(72) Inventors: Beatriz FERNÁNDEZ MUÑOZ, Sevilla (ES); Rosario SANCHEZ PERNAUTE, Sevilla (ES); Javier MARQUEZ RIVAS, Sevilla (ES); Elena GONZALEZ MUNOZ, Malaga (ES)

(21) Appl. No.: 17/059,083

(22) PCT Filed: May 28, 2019
(86) PCT No.: PCT/EP2019/063888

§ 371 Date: Nov. 25, 2020

(30) Foreign Application Priority Data
May 28, 2018 (EP) 18382367.3

(51) Int. Cl. C12N 5/0797

(52) U.S. Cl. CPC C12N 5/0623; C12N 2501/115; C12N 2533/90; C12N 2501/52; C12N 2533/52; C12N 2501/235

(57) ABSTRACT

The present invention provides a novel method to isolate and expand pure neural stem cells (NSCs) from cerebrospinal fluid (CSF) of premature babies with Intraventricular haemorrhage, which produces a population enriched in NSC-CSF cells free of contaminating fibroblasts and other cell types. The present invention also includes substantially pure populations of CSF-NSC cells, and their use to treat and prevent diseases and injuries, including Intraventricular haemorrhage and post-hemorrhage hydrocephalus/developmental deficits.

### FIELD OF THE INVENTION

The present invention provides a novel method to isolate and expand neural stem cells (NSCs) from cerebrospinal fluid (CSF) of premature babies with Intraventricular haemorrhage, which produces a population enriched in CSF-NSC cells free of contaminating fibroblasts and other cell types. The present invention also includes substantially pure populations of CSF-NSC cells, and their use to treat and prevent diseases and injuries, including Intraventricular haemorrhage and post-hemorrhage hydrocephalus.

### BACKGROUND OF THE INVENTION

Intraventricular haemorrhage (IVH) is a common cause of morbidity and mortality in premature infants. The incidence of premature infants with IVH has declined in recent years, but remains a significant problem in infants with very low birth weight (VLBW<1500 g) and extremely low birth weight (ELBW<1000 g). IVH is classified according to the degree of haemorrhage and subsequent ventricular dilatation (Grade I-II as defined moderate IVH, and grade III-IV as defined severe IVH) (Premature infants with severe IVH present higher risk to develop post-hemorrhage hydrocephalus (PHH) or periventricular leukomalacy, and exhibit long-term neurological deficits with cognitive and psychomotor disabilities. No cure for IVH has been developed so far.

Typically, IVH initiates in the subependymal germinal matrix, the source of cerebral neural stem cells during cortex development, between approximately the 10th and 24th gestational weeks (Ballabh, 2010. Pediatr. Res. 67, 1-8). In the haemorrhage stage, there is a rupture of the germinal matrix that entails loss of neural stem cells and disturbs the normal cytoarchitecture of the ventricular zone compromising the organization and function of the cerebral cortex (reviewed in Guerra, 2014. Fluids Barriers CNS 11, 1-10).

Neural stem cells (NSCs) have differentiation and self-renewing potential and express neuroprotective factors, capabilities that make them suitable to regenerate lost tissue having a great therapeutic potential for the treatment of different pathologies (Ludwig, 2018. Neural Regen. Res. 13, 7-18; Tang, 2017 Cell Death Dis. 8). This potential has been tested in preclinical studies showing some success in a variety of animal models of different nervous system diseases and in clinical trials for spinal cord injury, amyotrophic lateral sclerosis, glioma, cerebral palsy and other neurological disorders. Although data from many of these clinical trials are still being compiled, some improvements in neurological function has been reported and safety of the NSC-based therapies has been confirmed (reviewed in Tang, 2017). NSCs can be isolated from the central nervous system (CNS) of fetuses and adult tissue, but these procedures require human embryos or invasive procedures, respectively, which have obvious limitations. NSCs can be also derived from pluripotent stem cells and somatic cells through reprogramming protocols (Tang, 2017) but these are poorly standardized procedures and show low efficacy giving rise to a low purity NSCs population. Cerebrospinal fluid of spina bifida cases has been also recently proposed as a new source for NSC/NPCs. Nonetheless, NSCs from the CNS of fetuses remain the most used cell type for clinical use. Indeed, several companies are using this cell type in clinical trials for neurological disorders (reviewed in Tang, 2017). Despite the encouraging results of some of these clinical trials, the scarcity of source material and the ethical problems associated with isolation of the NSC is an obvious constraint for the use of these cells as a therapy to improve patient quality of life.

### BRIEF DESCRIPTION OF THE INVENTION

Here we demonstrate that a novel type of neural stem cells can be easily and robustly isolated from preterm infants with IVH. We have characterized the cell population obtained from the cerebrospinal fluid (CSF) and found that these cells are very similar to foetal forebrain NSCs, and not to other stem cell types, such as CD34 positive cord blood or bone marrow mesenchymal stem cells. However, these CSF-NSCs present several distinctive hallmarks such as ventral regional transcription factors and an increased expression of podocalyxin (PODXL) or IL1RAP. These CSF-NSCs are directly isolated from the liquid obtained from the cerebral ventricles during neuroendoscopic irrigation performed as treatment of posthemorragic hydrocephalus, and pose no ethical concerns as the fluid is usually discarded. CSF-NSCs could be useful for the development of autologous therapies for infants with IVH and PHH or perhaps for developing allogeneic therapies for different neurological disorders, and for furthering our understanding of human late brain development.

### BRIEF DESCRIPTION OF THE FIGURES

FIG. 1. Recovery of CSF and irrigation fluid from preterm infants with PHH. (A) Images of the surgical intervention by neuroendoscopy. (B) Information of PHH cases studied in this article. EGA=Estimated gestational age.

FIG. 2. —Isolation of NSC-like cells from CSF of PHH patients. (A) Phase contrast microphotographs of CSF-derived NSCs cultures at different days after isolation. Scale bar images: 100 μm. Cells from 8 donors have been processed so far. Representative pictures are shown. (B) Flow cytometry analysis of CD133, CD24, CD34 and CD45 of a representative sample. (C) Comparison of CD markers expression between early and late passage CSF-derived cells. There were no significant differences between early and late passage. Data are mean±SEM of 8 independent biological samples. (D) Expression of NSCs markers by CSF derived cells. CSF-NSCs were stained with anti-Sox1, Sox2, Ki67 and Nestin antibodies. Nuclei were stained with DAPI. At least 3 independent biological replicates have been processed. Representative confocal sections are shown. Scale bar for all images: 10 μm.

FIG. 3. —Gene expression profile of CSF-NSCs. (A) PCA analysis and Hierarchical clustering (B) of global gene-expression profiles. Venn diagrams showing differentially upregulated (C) and downregulated (D) genes between CSF and fetal NSCs. (E) Hierarchical clustering of CSF and fetal NSCs. (F) Examples of genes whose expression is differentially upregulated in CSF-NSCs.

FIG. 4. —CSF-NSCs present several distinctive hallmarks. (A) Expression of Podocalyxin, IL1RAP and MHC II in CSF-NSCs analysed by flow cytometry. Results are the mean of 3 independent biological replicates (B) Flow cytometry analysis for the expression of Podocalyxin, IL1RAP and MHC II of a representative sample. (C) Expression of PPAPDC1A, Frizzled-5, GPR50 and K2P10.1 (TREK-2) in CSF-NSCs. Nuclei were stained with DAPI. At least 3 independent biological replicates have been processed. Representative confocal sections are shown. Scale bar for all images: 10 μm.

FIG. 5. —CSF-NSCs maintain distinctive hallmarks after CD133+ MACS purification. (A) Cell morphology before and after CD133+ purification of a representative sample. (B) Expression of CD133 after MACS purification. Results are the mean of at least 3 independent biological replicates (C) Expression of Podocalyxin, IL1RAP and MHC II 2 passages after purification. Results are the mean of at least 3 independent biological replicates. (D) Dot plot comparison of global gene-expression profiles from non-purified and CD133+ purified NSCs. Differentially upregulated (purple) and downregulated (green) genes are showed. (E) Hierarchical clustering of global gene-expression profiles of non purified and CD133+ purified NSCs.

FIG. 6. —CSF-NSCs are genetically stable through passages. Karyotype analysis of two representative batches at passage 10.

FIG. 7. —Fibroblast-like cells from non-hemorrhagic CSF. (A) Representative phase contrast images of fibroblast-like cells obtained from non-hemorrhagic CSF (cases 14, 16, 17 and 18). Scale bar Passage 0 left=10 μm, Scale bar Passage 0 right and passage 1=100 μm (B) Immunofluorescence staining for fibroblast markers expression. Cells were stained with anti-CD13, Vimentin, Collagen I and Fibronectin antibodies. At least three independent biological replicates have been processed. Representative pictures are shown. Scale bar=10 μm.

FIG. 8. —CSF-NSCs are able to differentiate into neurons, oligodentrocytes and astrocytes. CSF-NSCs were cultured with 10% FBS medium to induce differentiation. Figure shows fluorescence images of CSF-NSCs derived cells expressing Dcx (neuronal lineage), GFAP (astrocyte lineage) and Olig2 (oligodendrocyte lineage).

FIG. 9. —CSF-NSCs do not express fibroblast markers. Fluorescence images of CSF-NSCs showing a positive staining for vimentin and a negative staining for CD13, Collagen 1 and Fibronectin fibroblasts markers.

FIG. 10. —CSF-NSCs are phenotypically stable through passages. Percentage of expression of NSCs markers at early and late passages. Data are mean±SEM of at least 3 independent biological replicates.

FIG. 11. —CSF-NSC and fetal-NSCs maintain stable gene-expression profiles through passages. Dot plot comparison of global gene-expression profiles from early and late passage of fetal (A) and CSF (B) derived NSC.

FIG. 12. —Isolation of NSC-like cells from the CSF of IVH patients. (A) Schematic representation of GM localization (in blue) around the ventricles (axial view), and computed tomography axial brain images depicting the bleeding area close to the head of the caudate nucleus and the presence of blood inside the ventricular system (blue arrows) in one of the cases. GM: germinal matrix LV: lateral ventricle. (B) Recovery of CSF and irrigation fluid from preterm infants with grade IV IVH. Images of the surgical intervention by neuroendoscopy: preparation (i); neuroendoscopic imaging of bleeding area before (ii) and after (iii) irrigation and before (iv) and after (v) sealing; collection of irrigation fluid (vi). (C) Phase contrast microphotographs of CSF-derived NSC cultures at different days in vitro (DIV) after isolation. Scale bar: 100 μm. (D) Proliferation was assessed by quantification of Ki-67 expression, which was not different between cultured cells at early (3) and late (7) passages. Scale bar 25 μm (insert: 7.5 μm). Representative confocal sections are shown. (E) Flow cytometry analysis of CD133, CD24, CD34 and CD45 at early and late passage. There were no significant differences between conditions. Data are mean±SEM of 7 independent biological samples (CD133). The 42-weeks-old case (pink symbols) was excluded from further analysis.

FIG. 13. —CSF-derived cells display NSC features. (A) Expression of NSC markers Sox2, Nestin, and BLBP (FABP7) was demonstrated by IF. (B) CSF-NSC cells showed neural tri-lineage differentiation when grown in 2% FBS medium for 2 weeks, generating doublecortin (DCX), β-III-tubulin (βIIItub), glial fibrillary acidic protein (GFAP) and Olig2 positive cells in the culture. Nuclei were counterstained with DAPI. Representative confocal images (maximum projections) of 3 independent biological samples and (C) corresponding quantification, shown as % over total cells. Scale bar: 50 μm.

FIG. 14. —CSF-derived cells display a GM-NSC gene expression profile. (A) PCA analysis of global gene-expression profiles. (B) Venn diagrams showing the number of genes differentially regulated in CSF-derived GM-NSC relative to other stem cell types (2-fold change, FDR p<0.05). (C) Volcano plot of genes differentially regulated in CSF-derived GM-NSC and fetal forebrain NSC. Highlighted are markers that identify regional populations including genes that have been previously associated with germinal zones and forebrain regionalization (see also schematic in D) and in bold putative candidates for prospective identification of GM-NSC. (D) Expression levels of NSC and regional forebrain markers. (E) Enrichment network analysis of upregulated genes in GM-NSC relative to fetal NSC, profiled across brain regions according to the Allen brain atlas, and schematic neuroanatomical representation on coronal brain sections showing their periventricular, subcortical location. (F) Expression levels of candidate genes that are differentially expressed and could identify the GM-NSC population. (G) Expression levels of selected genes by semi-cuantitative RT-PCR. A: anterior, P: posterior, D: dorsal and V: ventral. BNST: bed nuclei of the stria terminalis; CD: caudate nucleus; cGE: caudal ganglionic eminence; DThal, dorsal thalamus; GPe: external globus pallidus; GPe: internal globus pallidus; IMD: intermediodorsal; LGE: lateral ganglionic eminence; MGE: medial ganglionic eminence; MD: mediodorsal; Put: putamen; SVZ: subventricular zone; Sept: septum; Thal: thalamus; V Pall: ventral pallidum.

FIG. 15. —GM-NSC signature is maintained after CD133 sorting. (A) Cell morphology and expression of CD133 after MACS purification. Results are the mean of 3 independent biological replicates. Scale bar: 100 μm (B) PCA analysis of early, late and sorted GM-NSC populations. (C) Venn diagrams representing the transcriptional changes related to cell propagation (early vs late) and CD133 sorting (2-fold change, R ANOVA FDR F<0.05). There were no genes differentially expressed between early and late passages. Genes downregulated in the CD133 sorted cells corresponded to neuronal pathways and those upregulated are indicative of a less differentiated stage. (D) Expression of NSC markers and GM-NSC at the RNA level in early, late and CD133+ purified cells. There were significant changes in the expression of PARM1 and KCNK10. Flow cytometry analysis of Podocalyxin (E), IL1RAP (F) and MHC II expression (G) in a representative sample and the corresponding quantification of 3 independent biological replicates before and after MACS purification. Immunofluorescence analysis of the expression of PLPP4 (H), Frizzled-5 (I) and TREK-2 (J) before and after MACS purification. Representative confocal images (maximum projection of z-stacks) of at least 3 independent biological replicates. Scale bar: 25 μm.

FIG. 16. —CD133⁺ purified GM-NSC engraft into nude mice. Immunofluorescence analysis of the expression of HuN (green) to identify human grafted cells and (A) Ki-67, (B) SOX2, (C) β-III-tubulin and (D) GFAP (in red). Shown are representative confocal sections of 3 independent biological replicates and confocal z-stack orthogonal reconstructions to show co-localization.

FIG. 17. —CSF-derived cells are stable through passages. (A) GM-NSC phase contrast micrographs at early (0) and late (10) passage. Scale bar: 100 urn. (B) Growth kynetics of 3 representative lines showing cell number at each passage. (C) Karyotype analysis of a representative batch at passage 10.

FIG. 18. —Gating strategy and flow citometry analysis of lavage fraction and rebleeding samples. A) Gating strategy for CD133, CD34, CD24 and CD45 analysis by flow cytometry. The gating strategy is shown for one representative case (case 2 donor-derived cells). Cells were stained with isotype control (pink histogram) or with the corresponding antibody (blue histogram) as detailed in the experimental procedures section. Dead cells were exclude using propidium iodide (IP). B) Phase contrast images of CSF and lavage fractions 13 days after isolation and CD133, CD34, CD24 and CD45 analysis by flow cytometry of cells cultured from CSF and lavage fraction at passage 3.

FIG. 19. —Fibroblast-like cells from non-hemorrhagic CSF. (A) Gray-scale top panels: Representative phase contrast images of fibroblast-like cells obtained from samples of nonhemorrhagic CSF with large volumes. 1-2 days after extraction a heterogeneous cell population could be observed. Scale bar Passage 0 left=10 μm, Scale bar Passage 0 right and passage 1=100 μm. Colored panels: immunofluorescence staining for fibroblast markers expression. Cells were stained with anti-CD13, Vimentin, Collagen I and Fibronectin antibodies. At least three independent biological replicates have been processed. Representative confocal pictures (maximum projections) are shown. Scale bar=50 μm. (B) Non-hemorrhagic cebrospinal fluid samples.

FIG. 20. —MHCII expression during brain development. Neuroanatomical representation of HLA-DPA expression in the fetal brain (21 post conception weeks) according to the prenatal laser microdissection array data in the atlas of the Developing Human brain. Note the high expression in the ventricular zone (VZ) including lateral and caudal ganglionic eminences and somewhat lower in the subventricular zone (SVZ). Image credit Allen brain Atlas.

FIG. 21. —Expression of CD markers and PCA analysis of GM-NSCs after CD133+ purification. (A) Flow cytometry analysis of CD24, CD34 and CD45 after CD133+ purification. Data are mean±p SEM of 3 independent biological samples. (B) PCA analysis of global gene-expression profiles including early, late and CD133 purified GM-NSCs.

### LIST OF TABLES

Table 1. —Antibodies.

Table 2. —Identification of tissue transcriptional profiles corresponding to genes upregulated in CSF-NSCs with respect to fetal NSCs. Shown are the 5 top categories with the number of genes, percentages of the differentially expressed genes, significance and the list of genes. Note that genes can appear in several lists and some genes may not map to any of the archived tissues (EnRichR-ARCHS4 Tissue).

Table 3. —Identification of tissue transcriptional profiles corresponding to genes upregulated in fetal NSCs with respect to CSF-NSCs. FDR=0.01 N=3 biological samples at P3 and P7. Shown are the 5 top categories with the number of genes, percentages of the differentially expressed genes, significance and the list of genes. Note that genes can appear in several lists and some genes may not map to any of the archived tissues (EnRichR-ARCHS4 Tissue).

Table 4. Identification of tissue transcriptional profiles corresponding to genes differentially expressed in CSF-NSCs with respect to fetal NSCs. FDR=0.05 N=5 biological samples at P3 (EnRichR-ARCHS4 Tissue).

Table 5. Antibodies.

Table 6. Primers.

### DETAILED DESCRIPTION OF THE INVENTION

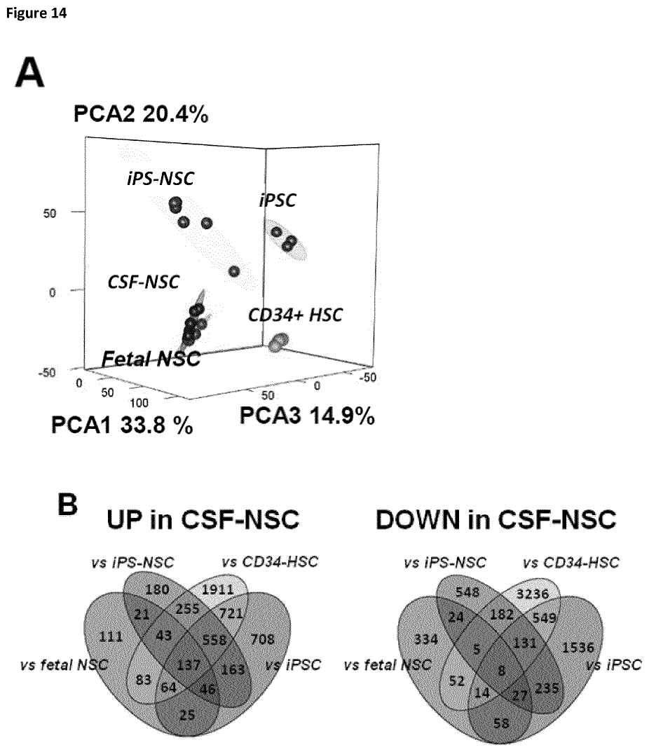

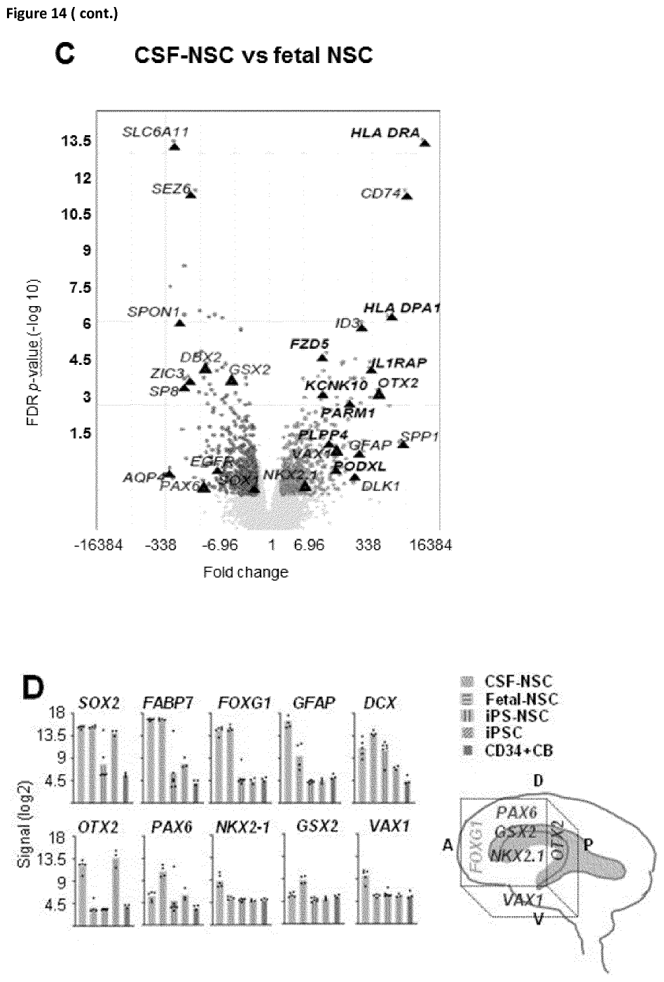

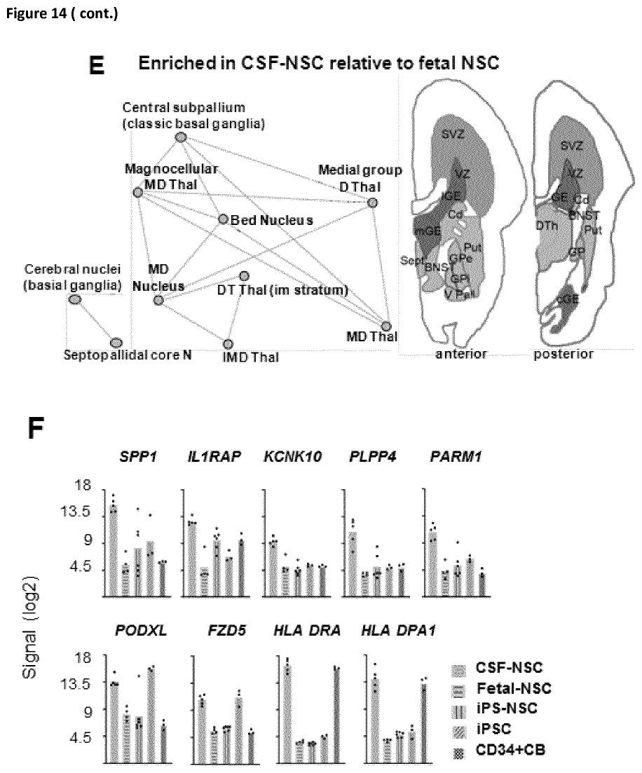

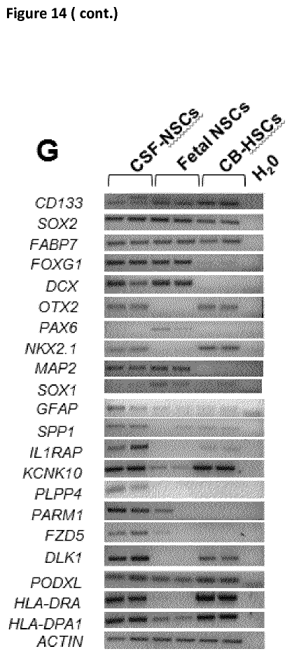

In the present invention, we demonstrate that premature babies with IVH are shedding NSCs from the germinal matrix into the CSF, a material that is regularly removed from these patients to ameliorate effects related to intracranial pressure and that is usually discarded in most of the hospitals. In this regard, the authors obtained from one batch approximately 3×10⁶ cells at passage 0. After 4 passages, they generated 250×10⁶ cells, a number we estimated enough for an autologous treatment, at least to put back in the germinal zone the NSCs that infants lose during IVH. In addition, we have demonstrated here that CSF-NSCs are phenotypically stable through passages and they adequately proliferate maintaining a stable karyotype, indicating that a large amount of stable cells can be obtained safely. Also, the transcriptomic profile suggested that GM-NSC were less committed than fetal NSC, which were derived from fetuses at earlier developmental stages (15-22 for fetal vs 26-36 weeks EGA in IVH). This is most likely related to a larger contribution of the cortical SVZ, which is greatly expanded in humans, than the VZ, to fetal dorsal forebrain volume (see schematic in FIG. 14D).

The expression arrays of stem cells obtained from CSF samples show that gene-expression profile is closer to foetal NSCs than to IPS-derived neural stem cells, bone marrow mesenchymal or cord blood hematopoietic stem cells. However, at FDR<0.05 and 2-fold change there are over 1000 genes differentially expressed in CSF-NSCs. Among those markers relatively overexpressed in the GM-NSC, FZDS, HLA related markers, Podocalyxin and IL1RAP are membrane-bound proteins that can be useful to isolate these cells.

We also show in this invention that CSF-NSCs cannot be obtained from non-hemorrhagic CSF. However, fibroblast-like cells can be isolated from these samples meaning that non-haemorrhagic CSF is a new source for fibroblast/mesenchymal stem cells isolation.

As used in the specification and appended claims, unless specified to the contrary, the following terms have the meaning indicated: A “stem cell” refers to an undifferentiated, multipotent, self-renewing, cell. A stem cell is able to divide and, under appropriate conditions, has self-renewal capability and can include in its progeny daughter cells that can terminally differentiate into any of a variety of different cell types. A stem cell is capable of self-maintenance, meaning that with each cell division, one daughter cell will also be on average a stem cell.

The non-stem cell progeny of a stem cell is typically referred to as “progenitor” cells, which are capable of giving rise to various cell types within one or more lineages. The term “progenitor cell” refers to an undifferentiated cell derived from a stem cell, and is not itself a stem cell. Some progenitor cells can produce progeny that are capable of differentiating into more than one cell type. A distinguishing feature of a progenitor cell is that, unlike a stem cell, it does not exhibit self-maintenance, and typically is thought to be committed to a particular path of differentiation and will, under appropriate conditions, eventually differentiate along this pathway.

The term “precursor cells” refers to the progeny of stem cells, and thus includes both progenitor cells and daughter stem cells.

Stem cells and progenitor cells derived from a particular tissue are referred to herein by reference to the tissue from which they were obtained. For example, stem cells and progenitor cells obtained from cerebrospinal fluid (CSF) of premature babies with Intraventricular haemorrhage are referred to as “NSC-CSF” or “CSF-NSCs” or germinal matrix-NSC.

A “clonogenic population” refers to a population of cells derived from the same stem cell. A clonogenic population may include stem cells, progenitor cells, precursor cells, and differentiated cells, or any combination thereof.

The term “purified” or “enriched” indicates that the cells are removed from their normal tissue environment and are present at a higher concentration as compared to the normal tissue environment. Accordingly, a “purified” or “enriched” cell population may further include cell types in addition to stem cells and progenitor cells and may include additional tissue components, and the term “purified” or “enriched” does not necessarily indicate the presence of only stem cells and progenitor cells.

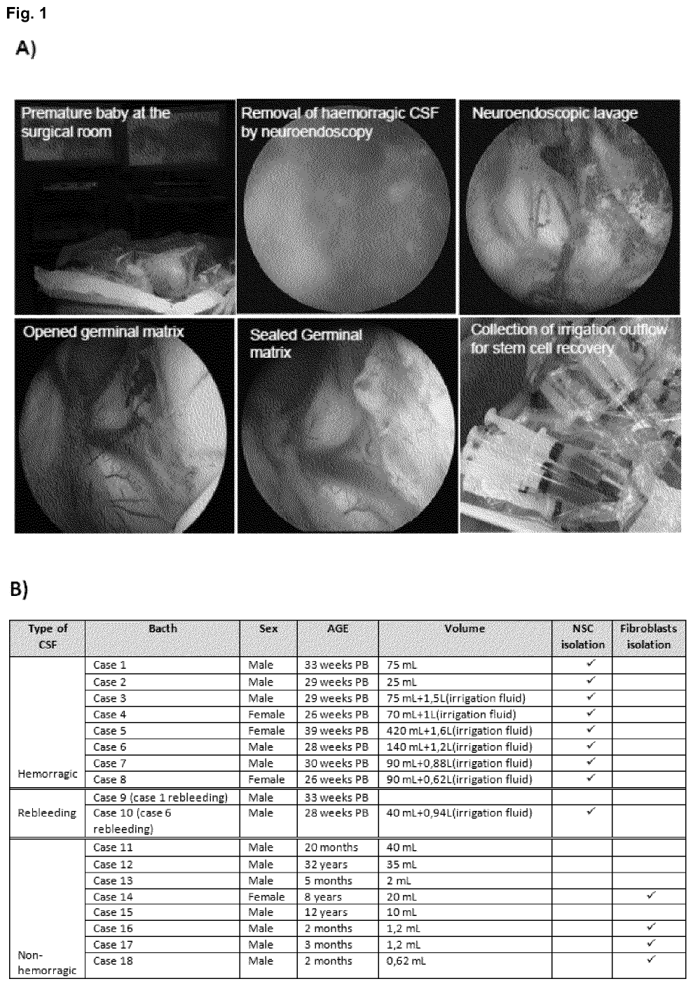

The present invention thus provides populations of cells enriched in stem cells obtained from cerebrospinal fluid (CSF) of premature babies with Intraventricular haemorrhage, which are preferably substantially free of contaminating fibroblasts and other cells. More particularly, these cells are preferably obtained from the ventricle of premature babies with the larger amount of hematoma, said ventricle is punctured with the surgical endoscope under intraoperative ultrasound guidance. When ventricular cavities are approached, under direct vision, continuous irrigation is established using warm lactate-free Ringer solution, by passive inflow via an infusion system through the irrigation channel of the endoscope. Simultaneously, a passive outflow is ensured through a second channel to balance the intracranial volume and avoid any significant changes in intracranial pressure. This outflow is preferably collected for subsequent recovery of stem cells through a three-way connection attached to syringes with preferably a luer-lock connection in order to assure sterility and minimal handling of haemorrhagic CSF. Irrigation is stopped once the fluid within the ventricular system is clear or hemodynamic instability appears during surgery. Typically, 1000-2000 ml of Ringer solution are used and collected in sterile syringes that are immediately closed with a cap to maintain a sterile liquid (FIG. 1A).

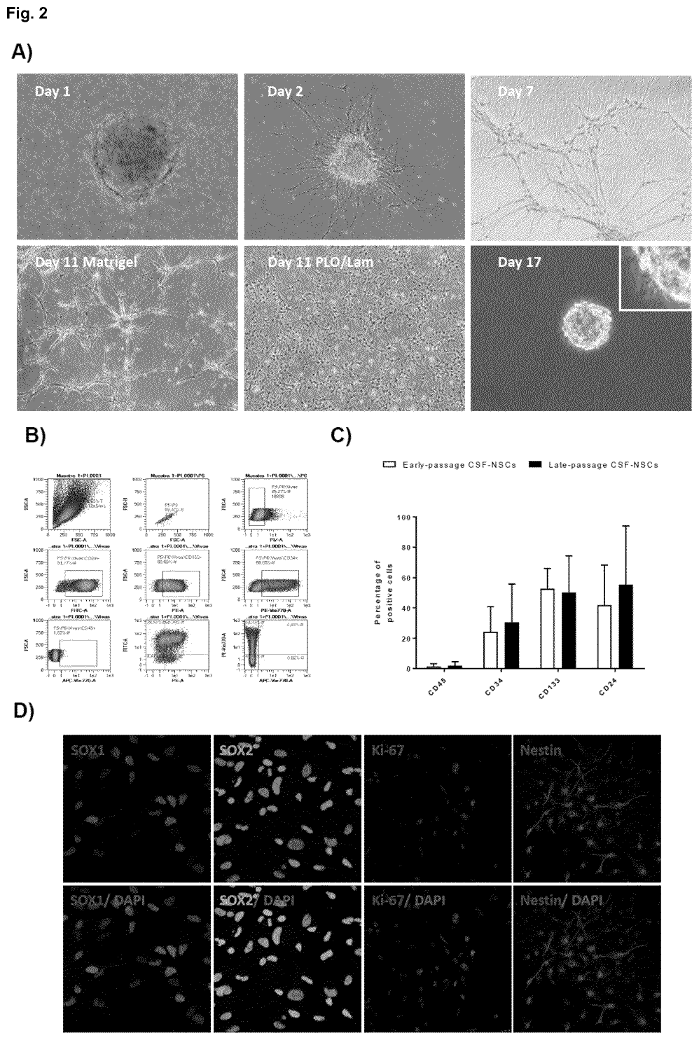

Preferably, after collection, CSF is centrifuged and a big red cell pellet is obtained. Cell suspension is then seeded in PLO-laminin or matrigel coated plates (see Experimental procedures for details of the protocol). Medium is preferably changed after 24-48 h and some groups of cells in the NSC-size and tissue spheres slightly adhered to the plate should be observed. 3-5 days after isolation some NSCs-like cells coming out from tissue spheres should be clearly detected. Around day 7 after isolation there should be some neurospheres in suspension and cells in adhesion starting to growth. Around day 8 after isolation cells should be passaged as neurospheres with accutase or in adhesion over matrigel or PLO/laminin coated plates (FIG. 2A). Cells are then expanded over more than 10 passages and they should show a doubling time of 5.5 days±3.28. The methodology detailed above, should obtained NSCs-like cells from sample of haemorrhagic CSF. It is noted that the present invention is not limited to the methods detailed above to obtain the populations of cells of the present invention.

At any rate, these populations, populations of cells enriched in CSF-NSCs obtained from cerebrospinal fluid (CSF) of premature babies with Intraventricular haemorrhage, are advantageous over previously described populations of purified stem cells and progenitor cells. In addition, these cell populations, preferably, do not include fibroblasts, which lead to undesired scar formation when administered to a wound or disease site. In addition, contaminating cells, such as fibroblasts, can proliferate more rapidly than stem cells and compete with stem cells in repopulating a tissue site when administered therapeutically.

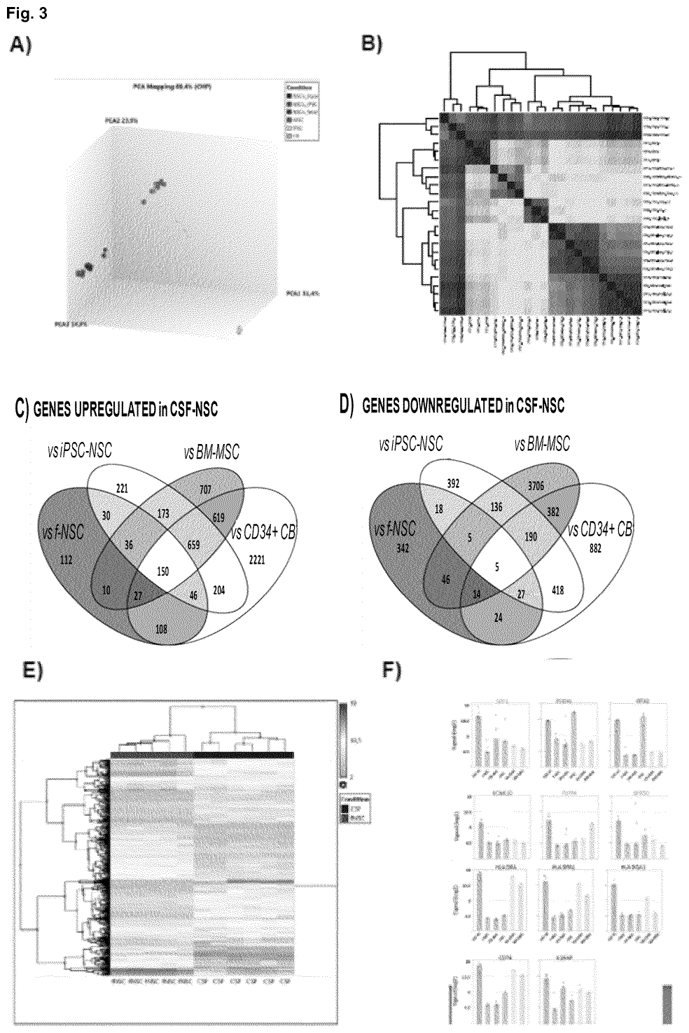

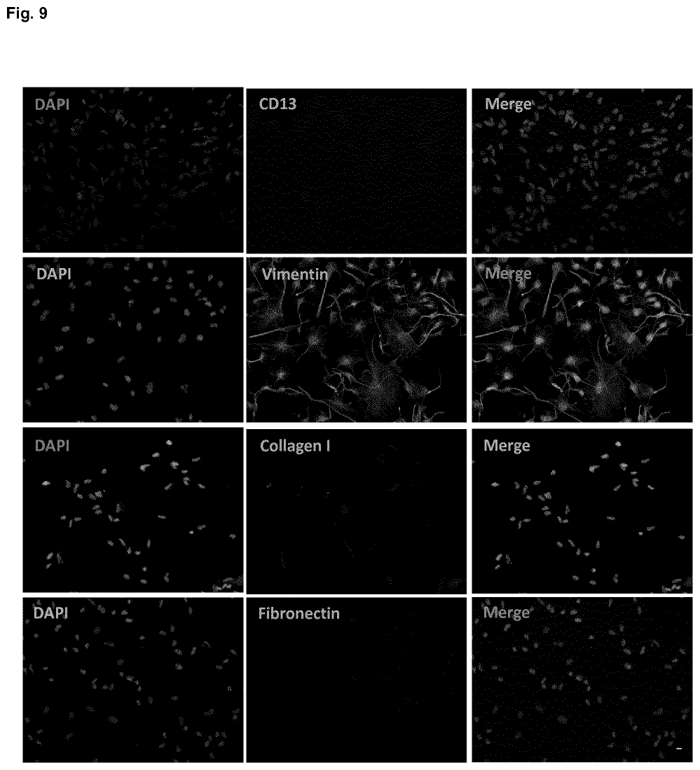

Thus, in various embodiments, an enriched cell population of the present invention comprises at least 40%, 50%, 75%, at least 80%, at least 85%, at least 90%, at least 95%, at least 96%, at least 97%, at least 98%, at least 99%, or 100% CD133 positive CSF-NSCs obtained from cerebrospinal fluid (CSF) of premature babies with Intraventricular haemorrhage, as indicated by the presence of one or more stem cell markers, such as CD133. In fact, such cell populations of the present invention, CSF-NSCs, are preferably positive for CD133, and optionally for CD34 and CD24, and negative for CD45 (FIGS. 2B and C). More preferably, the purified cell population of the present invention is further characterized by being positive by immunofluorescence to the expression of at least one or more of the following Sox1, Sox2, Ki67, Nestin and vimentin markers; and negative for fibroblast markers such as CD13, Collagen I and Fibronectin (FIGS. 2D and 9). Still more preferably, the purified cell population of the present invention is further characterized by overexpressing one or more, preferably all, of the following markers Podocalyxin, KCNK10 (K2P10.1), PLPP4 (PPAPDC1A), GPR50, HLA-DR-A, HLA-DP-A1, and IL1RAP in comparison to foetal NSCs; and by having downregulated the following genes TIAM1, EGFR, PAX6, AQP4 or GSX2 when compared with foetal NSCs. (FIG. 3, 4 and Table 2 and 3). The inventors report the expression of other GM-NSC markers that differentiate these cells from fetal NSC and were maintained after sorting CD133⁺ cells, such as PODXL, IL1RAP, HLA-DR, and FZDS, (but not TREK2, which was significantly decreased). These markers could therefore serve to isolate and identify human GM-NSC. PLPP4 expression was maintained but showed an intracellular, nucleolar localization.

In certain embodiments, the purified cell populations of the present invention are present within a composition, e.g., a pharmaceutical composition, adapted for and suitable for delivery to a patient, i.e., physiologically compatible. Accordingly, the present invention includes compositions comprising a stem cell population of the present invention and one or more of buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

In related embodiments, the present invention provides a pharmaceutical composition that comprises the purified cell populations provided herein and a biological compatible carrier or excipient, such as 5-azacytidine, cardiogenol C, or ascorbic acid.

In related embodiments, the purified cell populations are present within a composition adapted for or suitable for freezing or storage. For example, the composition may further comprise fetal bovine serum and/or dimethylsulfoxide (DMSO).

The present invention further provides methods of treating or preventing injuries and diseases or other conditions, comprising providing a cell population of the present invention, i.e., a population enriched in stem cells and progenitor cells, to a patient suffering from said injury, disease or condition. In particular embodiments, the cell population was generated using a tissue sample obtained from the patient being treated (i.e., autologous treatment). In other embodiments, the cell population was obtained from a donor, who may be related or unrelated to the patient (i.e., allogeneic treatment). The donor is usually of the same species as the patient, although it is possible that a donor is a different species (i.e., xenogeneic treatment).

In various embodiments, the stem cell populations and related compositions are used to treat a variety of different diseases, including but not limited to inflammatory diseases, demyelinating diseases, mental disorders, neurodegenerative diseases such as ELA, Alzheimer or Parkinson, neuromuscular diseases, and preferably for the treatment, preferably the autologous treatment, of premature babies having or suffering from Intraventricular haemorrhage or post-haemorrhage hydrocephalus.

In specific embodiments, the present invention provide a methods for treating or preventing premature babies having or suffering from Intraventricular haemorrhage or post-haemorrhage hydrocephalus. These methods comprise providing a cell population of the present invention, wherein said cell population is enriched in CSF-NSCs, to a patient diagnosed, suspected of having, or being at risk of Intraventricular haemorrhage or post-haemorrhage hydrocephalus. In a preferred embodiment, these are isolated from the patient being treated.

Cell populations and related compositions of the present invention may be provided to a patient by a variety of different means. In certain embodiments, they are provided locally, e.g., to a site of actual or potential injury or disease. In one embodiment, they are provided using a syringe to inject the compositions at a site of possible or actual injury or disease. In one embodiment, they are administered to the bloodstream intravenously or intra-arterially. The particular route of administration will depend, in large part, upon the location and nature of the disease or injury being treated or prevented.

Accordingly, the invention includes providing a cell population or composition of the invention via any known and available method or route, including but not limited to oral, parenteral, intravenous, intra-arterial, intranasal, intramuscular and intracranial injection or administration. Preferably, a cell population or composition of the invention is administered at caudate nucleus. The development of suitable dosing and treatment regimens for using the cell populations and compositions described herein in a variety of treatment regimens, including e.g., oral, parenteral, intravenous, intranasal, intramuscular and intracranial injection or administration and formulation, will again be driven in large part by the disease or injury being treated or prevented and the route of administration. The determination of suitable dosages and treatment regimens may be readily accomplished based upon information generally known in the art and obtained by a physician.

Treatment may comprise a single treatment or multiple treatments. In particular, for preventive purposes, it is contemplated in certain embodiments that purified cell populations of the invention are administered during or immediately following a stress that might potentially cause injury.

The present invention also provides kits useful in the preparation and/or use of the purified cell populations of the present invention, which are enriched in stem cells. For example, in one embodiment, a kit useful in the preparation of the purified cell populations is provided that comprises an agent that binds a cell surface marker of stem cells or progenitor cells, and conditioned medium. For example, a kit may include: a first container comprising an antibody specific for a stem cell surface marker, wherein said antibody is adapted for isolation or detection, e.g., by being conjugated to a fluorescent marker or magnetic bead; and a second container comprising conditioned medium. In various related embodiments, the kits may further comprise one or more additional reagents useful in the preparation of a cell population of the present invention, such as cell culture medium, and enzymes suitable for tissue processing. The kit may also include instructions regarding its use to purify and expand stem cells obtained from a tissue sample. In other embodiments, the kits may further comprise a means for obtaining a tissue sample from a patient or donor, and/or a container to hold the tissue sample obtained.

The following examples serve to illustrate but they do not limit the present invention.

### EXAMPLES

### Experimental Procedures

### CSF Collection

The study was approved by the Hospital Virgen del Rock) de Sevilla ethical comitee and CSF samples were obtained after parental informed consent. CSF samples were obtained from 27-36 weeks (EGA) preterm infants by neuroendoscopy at the Hospital Universitario Virgen del Rock) (Sevilla). The ventricle with the larger amount of hematoma was punctured with the surgical endoscope (AesculapMlnop™) under intraoperative ultrasound guidance. When ventricular cavities were approached, under direct vision continuous irrigation was established using warm lactate-free Ringer solution, by passive inflow via an infusion system through the irrigation channel of the endoscope. Simultaneously, a passive outflow was ensured through the second channel (1.4 mm wide) to balance the intracranial volume and avoid significant changes in intracranial pressure. This outflow was collected for subsequent recovery of cells through a three-way connection attached to 50 ml syringes with luer-lock connection in order to assure sterility and minimal handling of CSF. Irrigation was stopped once the fluid within the ventricular system was clear or at any time if hemodynamic instability appeared. Typically, 1000-2000 ml of Ringer solution were used and collected in 50 ml sterile syringes that were immediately closed to maintain sterility.

### CSF-NSCs Isolation

CSF samples were maintained at 4° C. until NSCs isolation. 2-24h after collection CSF was transferred to appropriate tubes and was centrifugated at 370 g for 10 min. Pellet was resuspended in 2 mL and the resulting supernatant after first centrifugation was centrifuged twice again obtaining more pellet. Cell suspension was counted and seeded in poli-L-ornithine Sigma/laminin from human placenta (Sigma) or hESC qualified matrigel (Corning) coated plates in NDMBL medium (DMEM/F12 (Thermo), 0.1 mM non-essential aminoacids (Sigma), 100 IU penicilin/100 μg/mL streptomycin (Sigma), 2 μg/mL heparin (Sigma) 1% N2 (Thermo), 1×B27 (Thermo), 20 ng/mL FGF (Miltenyi), 20 ng/mL EGF (Preprotech), 10 ng/mL LIF (Miltenyi)). Media was changed 24-48 h after seeding. Cells were seeded for expansion at 0.5×10⁶/mL cells in low binding flasks or at 12.000 cells/cm² in matrigel coated plates. Cells were expanded for 3 (early) and 7-10 (late) passages for characterization. Passage 7 was considered late-passage given that cells cannot be extensively expanded in a clinical setting. CD133 MACS sorting was performed using human CD133 microbeads (Miltenyi Biotec) following manufacturer instructions.

Fibroblasts Isolation from Non-Hemorrhagic CSF

Cells were cultured with a medium base of high glocuse DMEM supplemented with 10% FBS (Sigma), 0.1 mM non-esential aminoacids (Sigma), 100 IU penicilin/100 μg/mL streptomycin (Sigma) and 2 mM Glutamax (Thermo).

### Cell Samples

Human fetal NSC were derived from the forebrain of 15-22 weeks (EGA) fetuses that had undergone spontaneous in utero death (miscarriage). Tissue procurement was approved by the Ethics Committee of the Institute “Casa Sollievo della Sofferenza” after receiving the mother's informed, written consent. Fetal NSC lines have been extensively characterized (Mazzini et al., 2015 J. Tranl. Med. 13, 17; Vescovi et al., 1999 Exp. Neurol. 156, 71-83; Gelati et al. 2013. Methods Mol Biol. 2013; 1059:65-77).

The iPS lines used were generated in our lab from adult dermal fibroblasts by electroporation with episomal plasmids containing OCT4, SOX2, NANOG, KLF4, LIN28, c-MYC and SV40LT (MSU-EPI-hiPSC) or by transduction with retroviral vectors for the overexpression of OCT4, SOX2 and KLF4 (E1 L6-hiPSC). We also included CBiPS1sv-4F-5 derived from CD133+ umbilical cord cells by infection with sendai virus and embryonic stem cells WA09 (H9) (iPS cell lines, Cell Lines National Bank, http://www.eng.isciii.es).

For neural differentiation, iPS were cultured as embryoid bodies (EB) in TeSR2 medium (Stemcell Technologies) spiked with Rock inhibitor (Y-27632; 10 μM; Tocris Bioscience). After 7 days, EB were plated on matrigel and cultured in neural differentiation media. On day 10, retinoic acid (RA) was added to the medium. On day 15, neural tube-like rosettes were mechanically detached and cultured in neural differentiation media with FGF and EGF. Cells were expanded in suspension as neurospheres or in adhesion over matrigel during 6-7 passages before RNA extraction for transcriptomic analysis.

Umbilical CB samples were obtained from the Banc de Sang i Teixits, Barcelona. CD34+ cell purification was obtained as previously described (Giorgetti et al., 2009. Cell Stem Cell 5, 353-357). Briefly, mononuclear cells (MNC) were isolated from CB using Lympholyte-H (Cederlane) density gradient centrifugation. CD34⁺ cells were positively selected using Mini-Macs immunomagnetic separation system (Miltenyi Biotec). Purification efficiency was verified by flow cytometric analysis staining with CD34-phycoerythrin (PE; Miltenyi Biotec) antibody.

### Immunofluorescence

Immunofluorescence detection of proteins was performed in cells fixed with 4% paraformaldehyde in PBS and permeabilized with 0.1% TritonX-100. Primary and secondary antibodies used are shown in Table 1. Nuclei were stained with a 1 μg/ml solution of 4′,6-diamino-2-phenilindole (DAPI; Life technologies). Fluorescent microscopy was performed in a TiS microscope (Nikon) or in a Leica TCS-SP5. Images were assembled using Adobe® Photoshop® CS5.

### Flow Cytometry

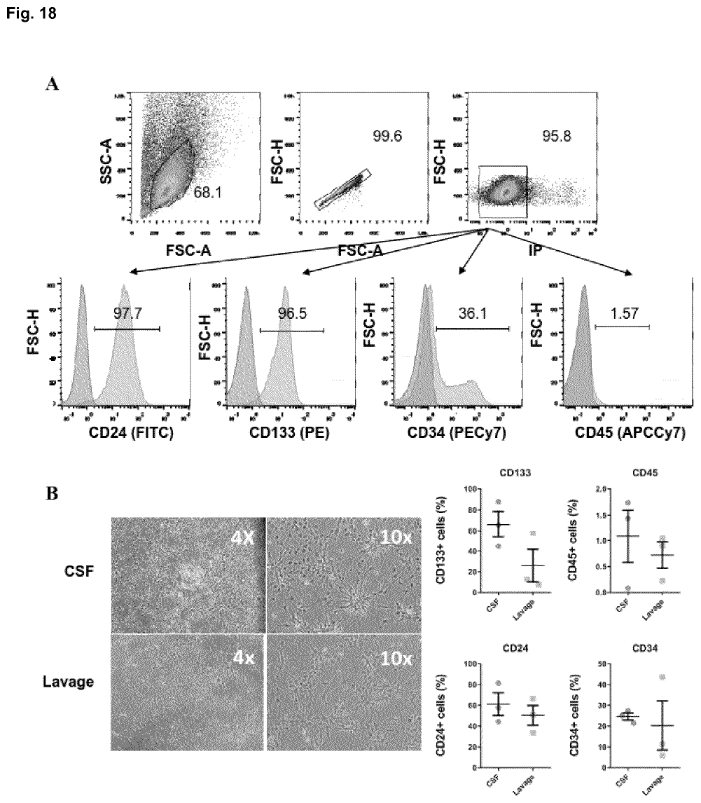

Live cells were incubated with primary antibodies (Table 1) for 30 min at 4° C. Fluorescence was estimated with a Macs Quant flow cytometer (Miltenyi) and results were analyzed with MacsQuantify 2.10 software and FloJ v10 software. IgG controls were always runned in parallel with samples. Gating strategy is shown in FIG. 18.

### Expression Arrays

RNA was extracted with RNeasy® Mini kit (Quiagen) following the instructions of the manufacturer. Samples were sent to the genomics unit of the Andalusian Molecular Biology and Regenerative Medicine Centre (CABIMER) for the quantification of RNA samples and execution of the expression arrays. RNA quality was analyzed by the Bioanalyzer 2100 (Agilent). Once RNA quality and quantity were confirmed, samples were labelled with biotin and hybridized with independent Human Clarion-S Arrays (Affymetrix). Samples were processed with Affymetrix GeneChip Scanner 7G, the fluidic station 450 of Affymetrix, and the obtained data were analysed with Affymetrix ° GeneChip® Command Console® 2.0 software and R. The microarray expression dataset is publicly available at the GEO repository (https://www.ncbi.nlm.nih.gov/geo/). Further analyses were performed using the Transcriptome Analysis Console (TAC, Affymetrix) v4.0 software and R version 3.5.0. Functional enrichment analysis was performed using the bioinformatics tool EnrichR (http://amp.pharm.mssm.edu/Enrichr/) (Chen et al., 2013 J. Eng. Res. 14; Kuleshov et al., 2016. Nucleic Acids Res. 44). Neuroanatomical references were obtained from the Allen Atlas of the developing human brain.

### Karyotyping

Cells seeded in matrigel-coated flasks to a cell density of 1,200,000 cells/flask were sent to the Biobank of the Andalusian Sanitary System, were G-banding Karyotyping was performed. Cells were treated with colcemid and potassium chloride and metaphases were treated with trypsin and stained with Giemsa to obtain the G-banding pattern. 15 metaphases were analyzed per cell line. The study was performed according to the International System for Human Cytogenomic Nomenclature (2016).

### Statistics

Data are presented as mean±s.e.m. Significance was determined using one-way analysis of variance (ANOVA) with a Bonferroni post-test or the Student's t-test. All statistical analyses were performed using GraphPad Prism 5.0 software and/or GraphPad Prism 8.01 software. Bioinformatic analyses were performed using Affymetrix and R software with t, ANOVA and repeated measures ANOVA tests and selected thresholds as indicated in the text and figures.

### RT-PCR

0.1 micrograms of RNA were used for cDNA synthesis using Oligo-dT, RNase OUT™ and SuperScript II Retrotranscriptase (Invitrogen). PCR products were obtained using 5 nanograms of cDNA and Mytaq Red™ DNA Polymerase (Bioline) following instructions of manufacturer. Oligonucleotides used for amplification are described in Table 5.

Transplantation into Nude Mice

Ten nude mice received a single injection of 300,000 CD133⁺ cells in the striatum. 3 animals were sacrificed at 3 weeks to assess survival and 7 animals at 6 months. Transplantation experiments and analysis were performed as previously described for fetal NSC (Mazzini et al., 2015. J. Transl. Med. 13, 17; Rosati et al., 2018. Cell Death Dis. 9, 937). Animal care and experimental procedures were conducted according to the current National and International Animal Ethics Guidelines and approved by the Italian Ministry of Health.

### Results

### 1. NSCs-Like Cells Populate Hemorrhagic CSF of IVH Patients

26-39 weeks-old premature infants diagnosed with IVH Grade IV according to Papille grading (Papile et al., 1978. J. Pediatr. 92, 529-534) and PHH were treated by endoscopy to seal the injured germinal matrix and to remove hemorrhagic CSF from ventricular cavities, as a measure to reduce the intracerebral pressure and decrease the burden of blood degradation products that may act against the subependymal periventricular area. Neuroendoscopic techniques seem to decrease the need for subsequent shunt procedures and have fewer complications such as infection and development of multi-loculated hydrocephalus. Previous studies suggested that early removal of intraventricular blood degradation products and residual hematoma via neuroendoscopic ventricular irrigation is feasible and safe. Neuroendoscopic lavage was performed following the technique reported by Schulz et al., 2014 disability (Schulz, 2014. J. Neurosurg. Pediatr. 13, 626-635) with some modifications.

The ventricle with the larger amount of hematoma was punctured with the surgical endoscope (Aesculap MInop™) under intraoperative ultrasound guidance. When ventricular cavities were approached, under direct vision continuous irrigation was established using warm lactate-free Ringer solution, by passive inflow via an infusion system through the irrigation channel of the endoscope. Simultaneously, a passive outflow was ensured through the second channel (1.4 mm wide) to balance the intracranial volume and avoid any significant changes in intracranial pressure. This outflow was collected for subsequent recovery of stem cells through a three-way connection attached to 50 ml syringes with luer-lock connection in order to assure sterility and minimal handling of hemorrhagic CSF. Irrigation was stopped once the fluid within the ventricular system was clear or hemodynamic instability appeared during surgery. Typically, 1000-2000 ml of Ringer solution were used and collected in the 50 ml sterile syringes that were immediately closed with a cap to maintain a sterile liquid. Bleeding area was sealed with gelatin beads with thrombin (Floseal, Baxter Healthcare Corporation) (FIG. 1A). A total of 8 samples of hemorrhagic CSF of IVH patients and 2 rebleedings have been taken so far by this procedure (FIG. 1B). The clinical follow-up of donor infants has not identified, to date, any other pathology except the one secondary to IVH.

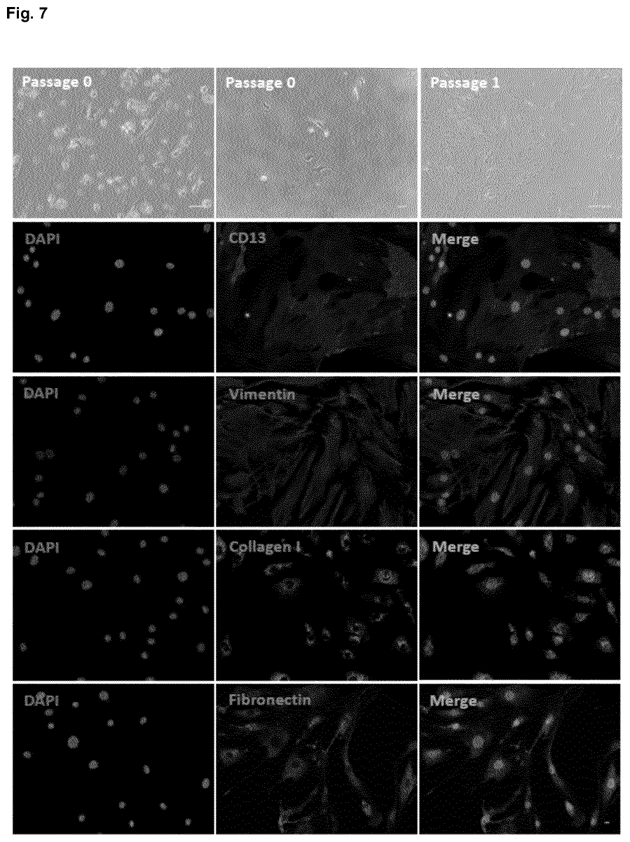

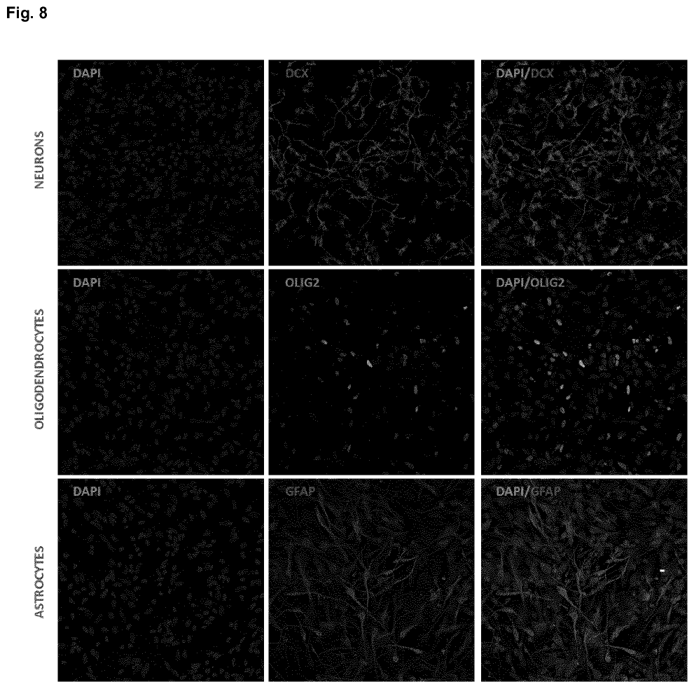

CSF was centrifuged and the obtained cell suspension was seeded in PLO-laminin or matrigel coated plates (see Experimental procedures for details of the protocol). 24-48 h after seeding the plate was filled of erythrocytes and blood cells in suspension but some groups of cells in the NSC-size and tissue spheres slightly adhered to the plate were observed (FIG. 2A). 2-5 days after isolation some NSCs-like cells coming out from tissue spheres were clearly detected. Around day 7 after isolation we found some neurospheres in suspension and cells in adhesion starting to growth. Around day 8 after isolation cells were passaged as neurospheres with accutase or in adhesion over matrigel or PLO/laminin coated plates showing different morphology depending on the coating. Cells were expanded over more than 10 passages showing a doubling time of 5.5 days±3.28 and maintaining a normal and stable morphology and karyotype (FIG. 6). We obtained these NSCs-like cells from every sample of hemorrhagic CSF from PHH patients.

We tried to isolate these NSCs-like cells from samples of non-hemorrhagic cebrospinal fluid and rebleedings of PHH patients (see FIG. 1B for details about cases) with the same protocol described before. 1-2 days after extraction a heterogeneous cell population could be observed (FIG. 7). However, we did not isolate neural progenitor-like cells from none of these non-hemorrhagic CSF samples with the exception of one of the rebleedings (case 10). In turn, we were able to growth fibroblast/mesenchymal-like adhered cells in samples with volumes higher that 20 mL when media was changed by fibroblast-specific media (cases 14, 16, 17 and 18). These cells were positive for CD13, vimentin, collagen I and Fibronectin fibroblast markers (FIG. 7) and negative for Sox1 and Pax6 NSCs markers.

The neural progenitor-like cells obtained from hemorrhagic CSF (CSF-NSCs) were analysed by flow cytometry at early (passage 3) and late passage (passage 7). Samples were positive for CD133 and CD24, and negative for CD45, a similar pattern to that published for foetal NSCs, however, CSF-NSCs showed a higher percentage of CD34 positive cells (FIGS. 2B and 2C).

CSF-NSCs were analyzed as well by inmunofluorescence for the expression of NSCs (Sox1, Sox2, and Nestin) and fibroblasts (CD13, Collagen I, vimentin and Fibronectin) markers. Cells were positive for all the markers analysed with the exception of fibroblast markers CD13, Collagen I and Fibronectin (FIGS. 2D and 9). Cells were also positive for Ki67 (FIG. 2D), indicating that they are proliferative cells. In order to test whether CSF-NSCs are tripotent stem cells they were cultured in medium with 10% FBS. After two weeks, CSF-NSCs derived cells expressed markers of neurons (Dcx), astrocytes (GFAP) and oligodendrocytes (Olig2) (FIG. 8). These data suggested that hemorrhagic cebrospinal fluid-derived cells are neural stem cells and that there is not contamination with fibroblast/mesenchymal cells.

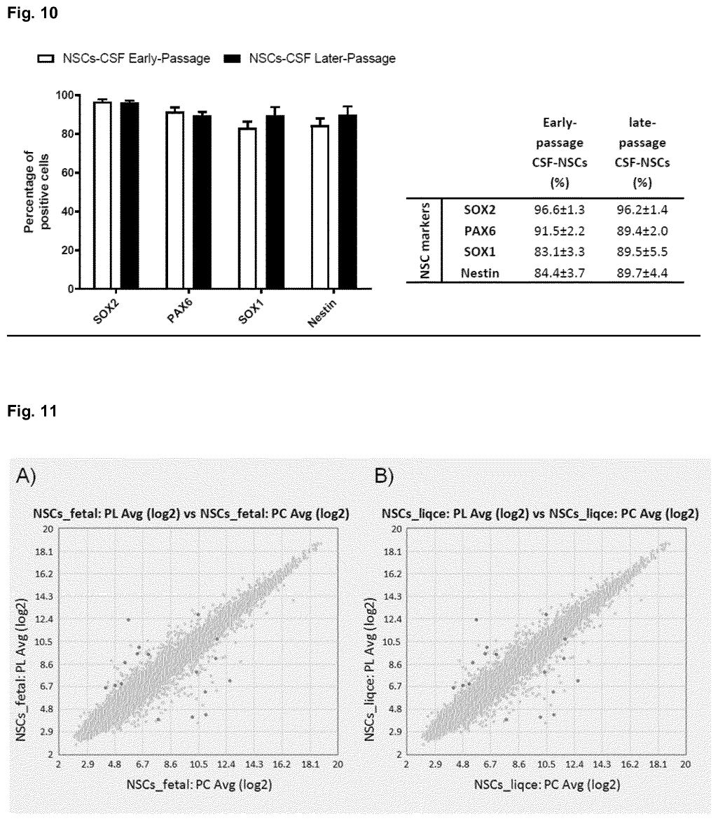

There were no significant differences in marker expression between early and late passage CSF-NSCs (FIGS. 2B and 10) indicating that these NSCs maintain a stable inmunophenotype for at least 7 passages.

Eight consecutive cases with a clinical and radiological diagnosis of grade IV IVH (Table 1) underwent a ventricular neuroendoscopy to seal the bleeding GM and remove the hemorrhagic CSF from the ventricular cavities (FIG. 12A-B). Neuroendoscopic lavage was performed following the technique reported by Schulz et al., 2014 (Schulz et al., 2014. J. Neurosurg. Pediatr. 13, 626-635) with few modifications. Typically, 1000-2000 ml of Ringer solution were used and collected in 50 ml sterile syringes.

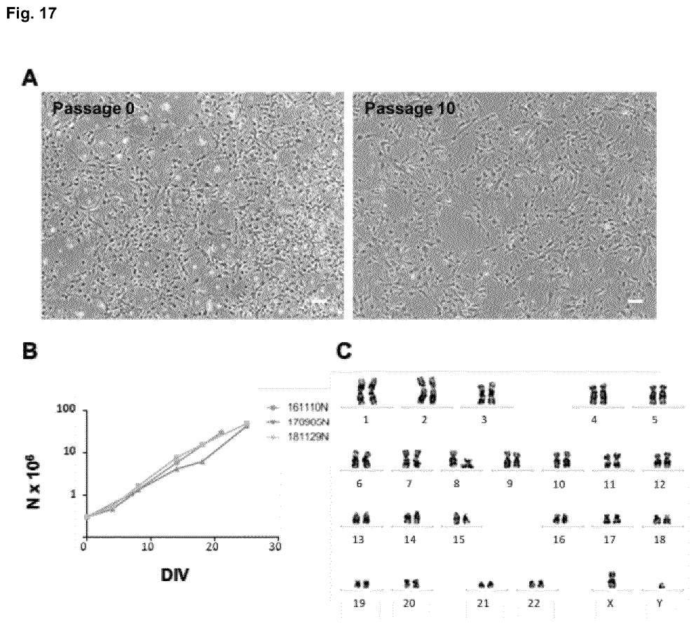

After centrifugation, the cell pellet was seeded on poly-L-ornithine/laminin (POL) or matrigel coated plates and cultured in an N2/B27 serum free medium with mitogens. 24-48 h after seeding, small aggregates were observed amidst abundant erythrocytes and blood cells in suspension (FIG. 12C). Cells were enzymatically dissociated and passaged as neurospheres or in adhesion (FIG. 12C) for more than 10 passages showing a doubling time of 4.86±0.97 days and maintaining a stable morphology and karyotype (FIG. 17). Quantification of Ki-67 showed no significant decrease in proliferation between early and late passages (FIG. 12D). Likewise, expression of the stem cell marker prominin-1 (CD133) was maintained through passages (FIGS. 12E and 18). An exception was the 42-weeks-old sample (FIG. 12E, pink symbols) in which the percentage of CD133⁺ cells dropped drastically upon passaging. This case was excluded from further analyses given that IVH in full-term neonates most often originates in the choroid plexus (Inder et al., 2018. Preterm Intraventricular Hemorrhage/Posthemorrhagic hydrocephalus. In: Volpe's Neurology of the Newborn, 6th, Volpe JJ (Ed), Elsevier, Philadelphia 2018. p. 637).

We next analyzed whether the cell population obtained from hemorrhagic CSF had a similar expression pattern of cluster of differentiation (CD) surface antigens than that described for fetal NSC (Tamaki et al., 2002 J. Neurosci. Res. 69, 976-986; Uchida et al., 2000. Proc Natl Acad Sci USA. December 19; 97(26):14720-5). Like fetal NSC, most cells in CSF samples were positive for CD133 and all were negative for CD45, displaying a variable expression of CD24 (FIGS. 12E and 18). Intriguingly, some samples contained a substantial percentage of CD34 positive cells (FIGS. 12E and 18) which is not expressed by fetal NSC (Uchida et al., 2000. Proc Natl Acad Sci USA. December 19; 97(26):14720-5).

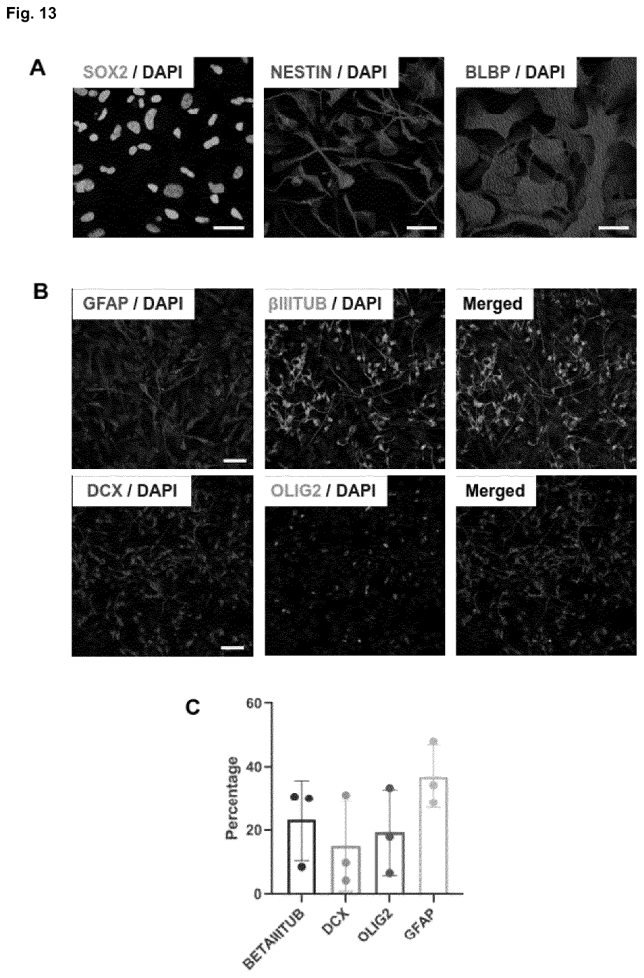

We next confirmed, by immunofluorescence, the expression of radial glia stem cell markers such as SOX2, nestin and brain lipid binding protein (BLBP, FABP7) (FIG. 13A). Finally, like NSC, our cultured cells showed in vitro trilineage differentiation potential, upregulating neuronal, astrocyte and oligodendrocyte cell markers upon withdrawal of mitogens and exposure to serum (FIG. 13B).

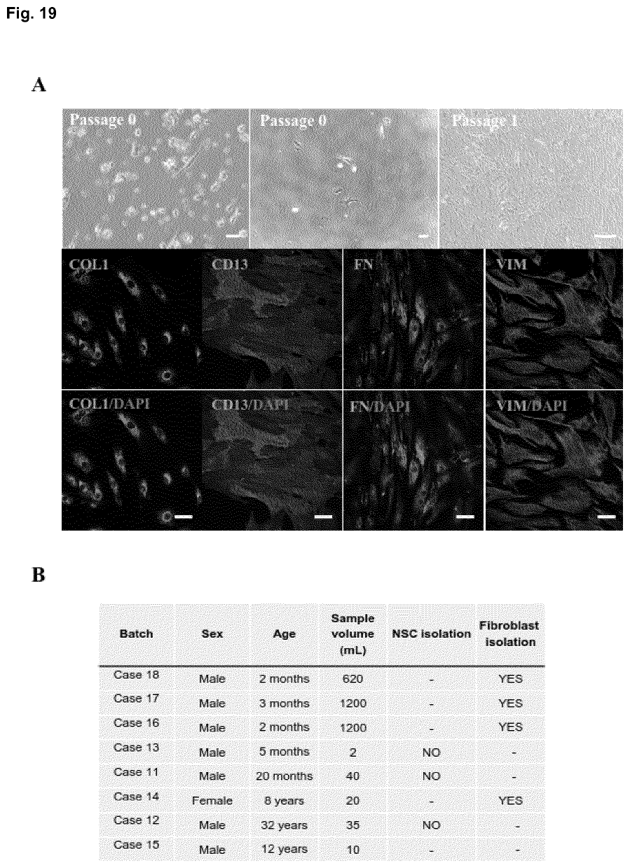

We did not obtain NSC-like cells from non-hemorrhagic CSF samples, although in some cases, changing to a serum-based media allowed us to grow fibroblast-like, adherent cells from samples with large volumes (>20 mL) (FIG. 19).

Taken together these experiments confirm that we can isolate NSC from the hemorrhagic CSF of preterm neonates.

### 2. Hemorrhagic Cebrospinal Fluid-Derived Cells are Similar to Foetal NSCs but Still Presenting Several Distinctive Hallmarks

To study whether CSF-NSCs were the result of a in vitro-forced differentiation of MSCs or HSCs or on the other hand were NSCs derived from the germinal matrix of PHH patients, we compared CSF-NSCs by transcriptome analyses with bone-marrow derived mesenchymal stem cells (BM-MSCs), cord blood CD34+ cells, iPS derived NSCs and fetal NSCs.

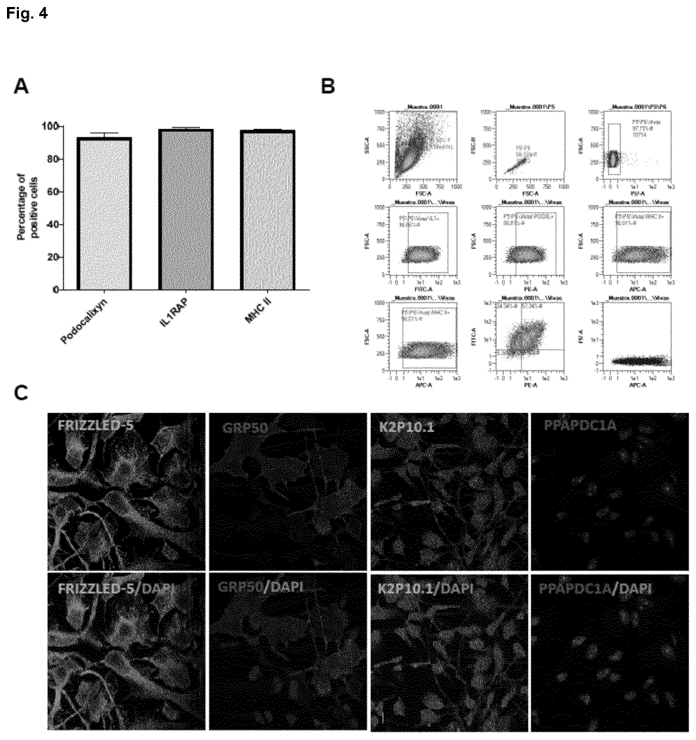

PCA mapping and hierarchical clustering of global gene-expression profiles showed that CSF-NSCs are clustered together with fetal NSCs being farther away from IPS-derived NSCs and stem cells from other sources (FIGS. 3A and 3B). However, we found 454 genes differentially expressed between CSF-NSCs and fetal NSCs indicating that there are some differences between them. 112 genes were differentially upregulated in CSF-NSCs versus foetal NSCs (FIG. 3C) and 342 differentially downregulated (FIG. 3D). Podocalyxin, KCNK10, PLPP4, GPR50, HLA-DR-A, HLA-DP-A1, HLA-DQ-A1 and IL1RAP were some of the genes overexpressed in CSF-NSCs (Table 2). This overexpression was validated by Flow Cytometry (FIGS. 4A and 4B) or inmunofluorescence (FIG. 4C). FGF11, TIAM1, EGFR, NCAM2, ADAMTS4 and ADAM19 were some of the downregulated genes when compared with fetal NSCs (Table 3).

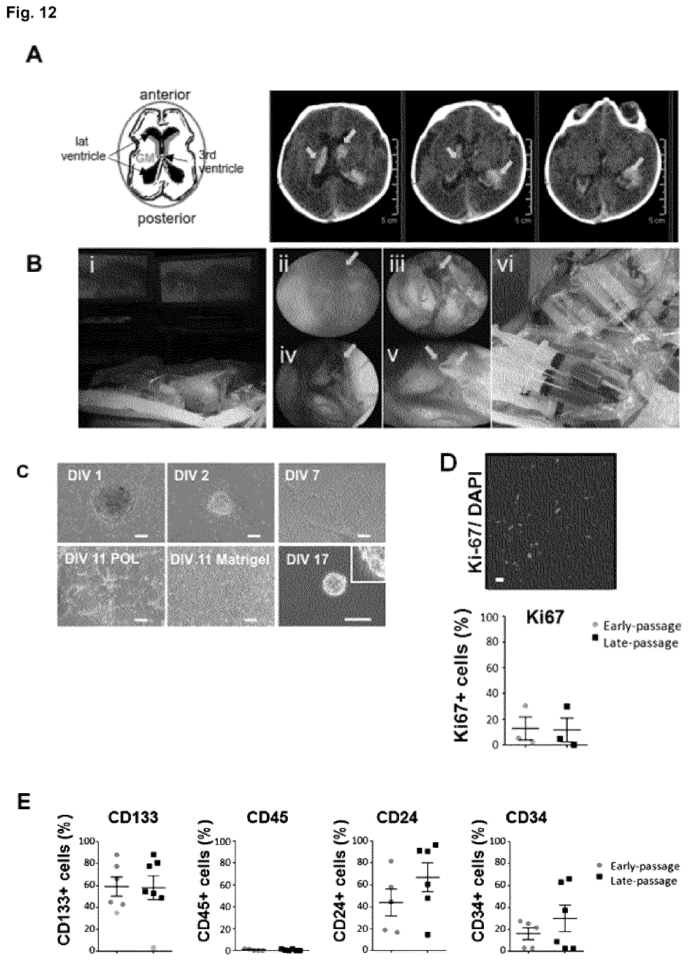

To study in more detail the dynamics of CSF-NSCs in culture, we performed comparative transcriptome analyses of 3 samples of CSF-NSCs at early and late passage finding no significant changes (FDR) in RNA expression between short and long passage in CSF-NSCs nor fetal NSCs (FIG. 11) indicating again that cells are phenotypically stable through passages.

We next performed a transcriptomic analysis to study the differences and similarities between NSC isolated from hemorrhagic CSF, fetal forebrain NSC and NSC derived from iPSC. Given that CSF samples contained mostly blood cells we also included in the analysis hematopoietic stem cells (CD34⁺ CB-HSC). Principal component analysis (PCA) mapping and hierarchical clustering of global gene-expression profiles showed that CSF-derived NSC clustered together with fetal NSC, being farther away from iPS-derived NSC (FIG. 14A). Pairwise comparisons showed the overlap in the expression profiles of the 3 types of NSC (FIG. 14B). Notwithstanding, there were 1061 genes differentially expressed between CSF-derived NSC and fetal NSC (using a false discovery rate (FDR) p value<0.05 and ±2 fold change) (FIG. 14C). Interestingly, enrichment analysis showed that the genes upregulated in CSF-derived NSC relative to fetal NSC mapped to the ventral forebrain periventricular nuclei-basal ganglia, thalamic and septal nuclei (FIG. 14D). This regional topography corresponds to the anatomical structures surrounding the GEs, most often affected by IVH.

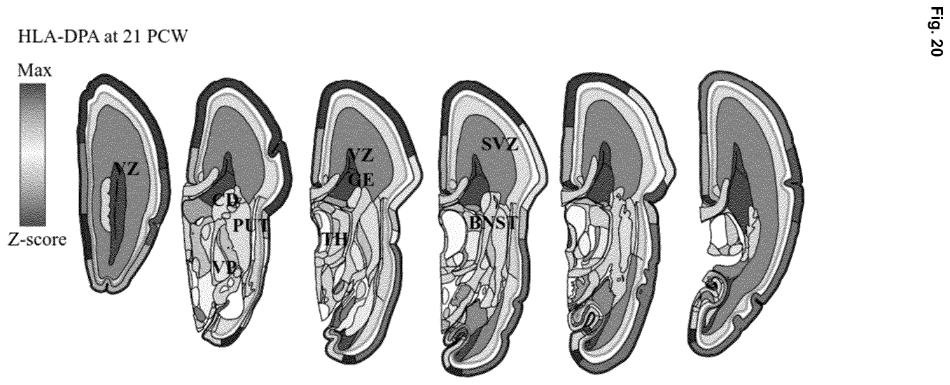

Consistent with our initial characterization, expression of radial glia and neural progenitor markers, such as SOX2, FABP7, FOXG1, DCX or SOX1 was similar in fetal and CSF-derived NSC (FIG. 14E). Glial fibrillary acidic protein (GFAP) was highly expressed in both types, but significantly higher in the CSF-derived NSC. GFAP expression is restricted to the VZ during primate brain development (Levitt and Rakic, 1980; Miller et al., 2014). Likewise, other transcripts enriched in human VZ relative to the SVZ, (the secondary proliferative area) such as SPP1, DLK1, IL1RAP (Fietz et al., 2012) or ID3, a marker of quiescent NSC, were also higher in CSF than in fetal NSC. On the other hand, expression of EGFR—that marks NSC activation—, AQP4, regulators of lineage commitment, such as SP8 or ZIC3, as well as more mature neuronal markers like MAP2 or MAPT was higher in fetal forebrain NSC (FIG. 14C). There were also substantial differences in the expression of regional transcription factors, with high expression of ventral and posterior such as OTX2, NKX2.1, VAX1 or LMO1, and lower of dorsal PAX6 and GSX2. In addition we identified several markers with putative membrane localization like KCNK10, PLPP4, IL1RAP, FZDS, MHCII and PODXL, and/or others, that could provide a distinctive signature for prospective isolation of GM NSC population (FIGS. 14C and 14F). Among those there was a remarkable upregulation of genes related to antigen presentation and immune response, in particular pertaining to the major histocompatibility complex II (MHCII) (FIG. 14F) which according to the developmental human brain atlas are highly expressed in the germinal zones during mid-gestational stages (www.brainspan.org) (Miller et al., 2014. Nature 508, 199-206) (FIG. 20). Differential expression of several of these markers was validated by PCR (FIG. 14F). This transcriptional profile is consistent with a ventral forebrain, GM origin of the cells isolated from the CSF and therefore we named them GM-NSC.

3. Purification of CD133+ Cells from Hemorrhagic Cebrospinal Fluid-Derived NSCs

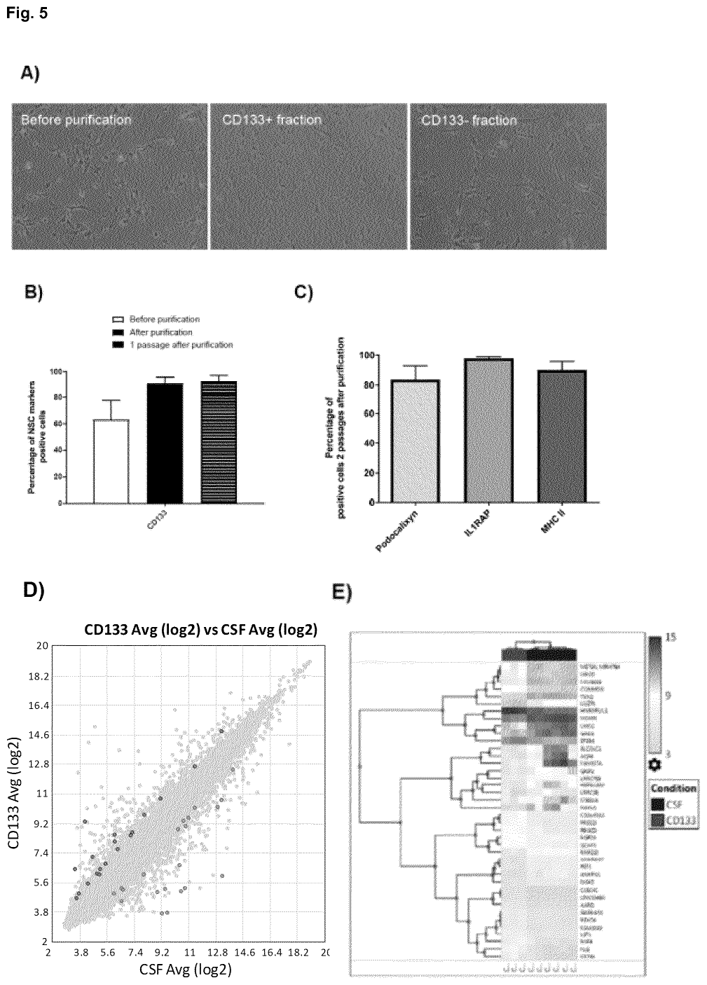

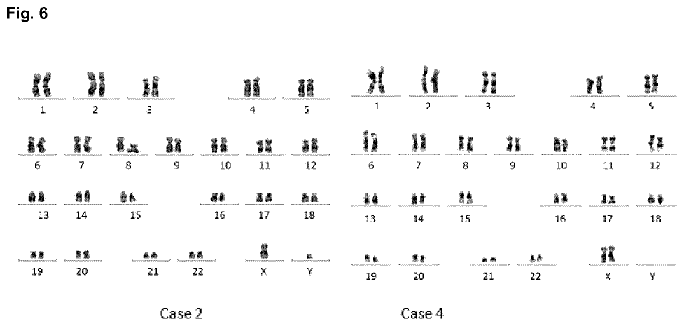

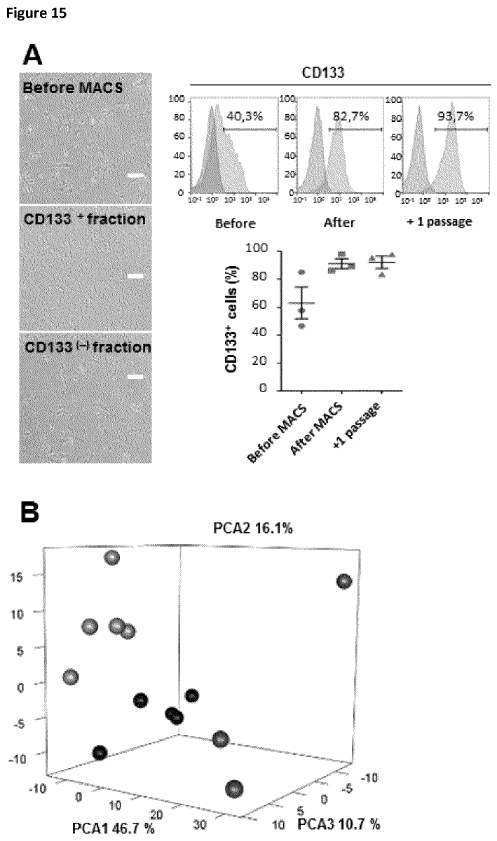

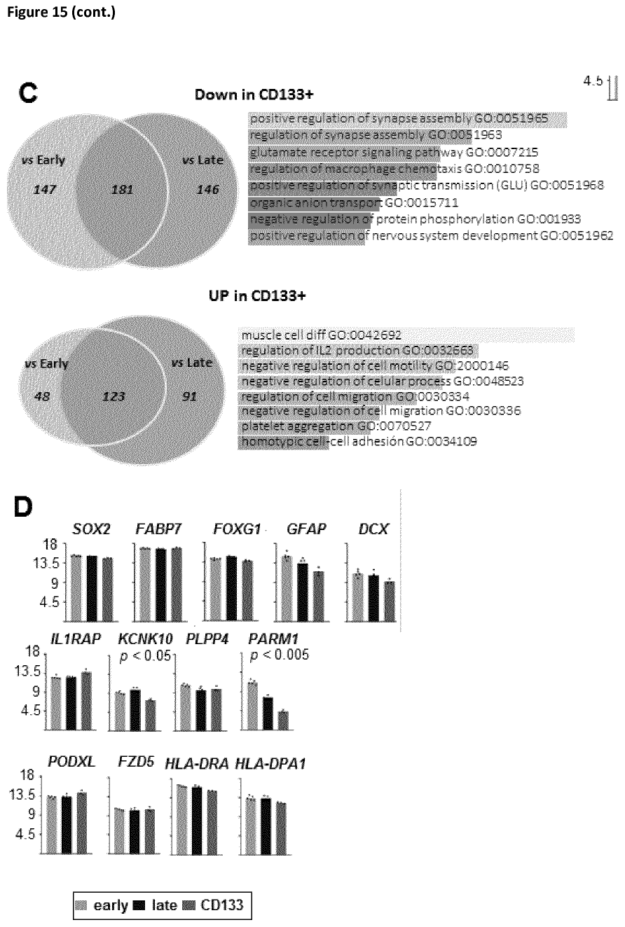

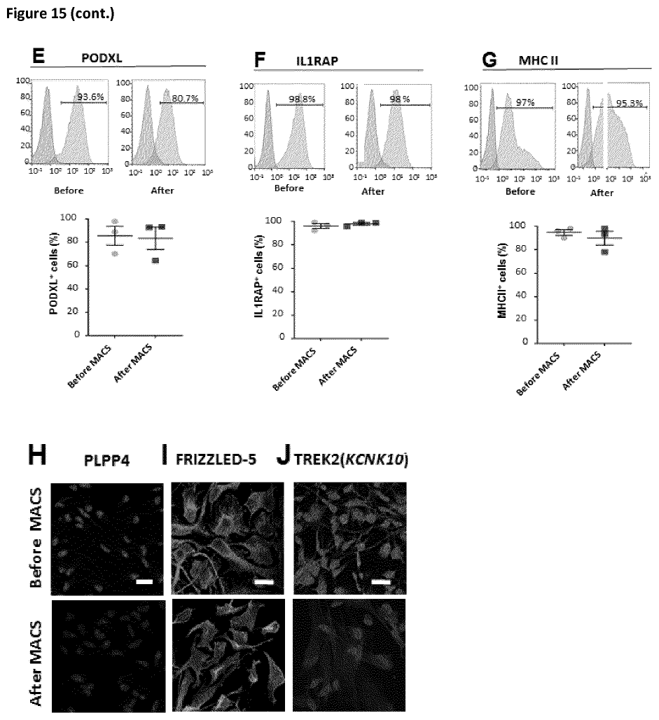

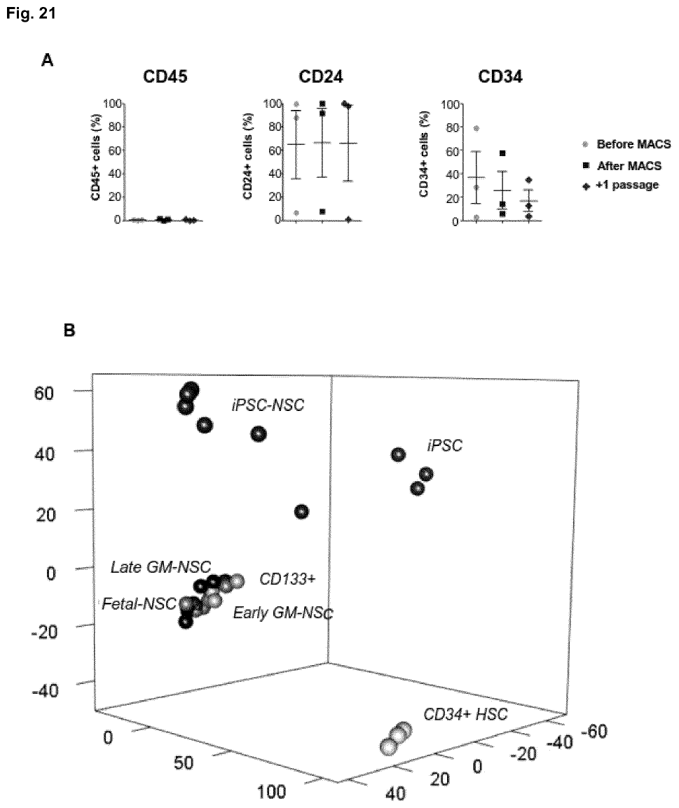

To assure NSC purity, CSF-NSCs were screened by magnetic activated cell sorting (MACS) with CD133 beads isolating a population 91.17+/−4.54% CD133+ that still maintain normal morphology (FIG. 5A) and Podocalyxin, IL1RAP and MHC II overexpression. These characteristics are stable for at least 2 passages (FIGS. 5B and C). High throughput transcriptome analysis confirmed that CD133+ cells maintain most of the distinctive CSF-NSC markers. As shown in FIG. 5D, few genes are differentially expressed when comparing non purified CSF-NSC and CD133+ MACS purified CSF-NSCs. As expected, differentially downregulated genes were related to differentiated lineages (FIG. 5E). Cells initially obtained from hemorrhagic CSF samples are a heterogeneous mixture of cellular types at different developmental and maturation stages, in particular taking into account that all these cases had parenchymal involvement (grade IV). Therefore, in order to better define putative GM-NSC specific features and obtain a more homogenous population for future in vivo applications, we selected CD133⁺ cells by magnetic activated cell sorting (MACS). Following MACS purification, we could expand and cryopreserve the cells, which maintained their typical morphology and the expression of CD133 (FIG. 15A). The CD45⁻, CD24⁺, CD34⁺ expression pattern did not significantly change although there was a trend to a decrease in CD34⁺ cells (FIG. 21).

We examined transcriptomic changes related to propagation and CD133 purification (FIG. 15C, D). Overall, only minor changes were observed during propagation. PARM1 was the only gene that dropped with passaging so that it was no longer different from fetal NSC. This gene is expressed in a subtype of GABA-vasoactive intestinal peptide (VIP) interneurons derived from the medial GE, suggesting that at least some differentiated cells are lost upon passaging. Similarly, upon purification, expression of genes related to more differentiated subtypes (corresponding to both regional and neural progenitors) was decreased (FIG. 15C). On the other hand, CD133⁺ sorted cells showed a relative enrichment in genes expressed at less differentiated stages. We examined the expression at the RNA level of the genes that could constitute a distinctive signature for GM-NSCs comparing early, late and CD133⁺ sorted GM-NSC (ANOVA, FDR F<0.05). Transcriptomic analysis confirmed that CD133⁺ cells maintained the expression of NSC and neural progenitor markers (FIG. 15D) and of most of the putative membrane markers that we had preselected, including genes related to antigen presentation. KCNK10 was significantly decreased but still significantly higher than in fetal NSC.

We validated the expression of these six candidate genes at the protein level using flow cytometry or immunofluorescence before and after MACS purification.

PODXL is an interesting glycoprotein involved in apical polarity, that belongs to the CD34 family of sialomucins, whose absence has been reported to cause ventricular enlargement in mice (Nowakowski et al., 2010. Mol. Cell. Neurosci. 43, 90-97). PODXL was expressed by nearly all cells in all samples and expression was maintained after CD133 sorting. The interleukin 1 receptor accessory protein (IL1RAP) is differentially expressed in the human VZ (Fietz et al., 2012. Proc. Natl. Acad. Sci. U.S.A. 109, 11836-11841). We confirmed IL1RAP expression in GM-NSC at the protein level by flow cytometry before and after MACS purification (FIG. 4F). Flow cytometry analysis was also used to confirm the expression of HLA-II at the protein level and appropriate membrane localization. Using a pan-antibody recognizing HLA-DRA, -DP and DQ (MHC II antibody), we confirmed that GM-NSC expressed high levels of MHC II receptors at the plasma membrane and that this expression was not significantly decreased after sorting the cells for CD133+ expression (FIG. 4G).

We used immunofluorescence to study the expression of PLPP4, a poorly characterized phospholipid phosphatase expressed in the brain (Human protein atlas). Despite its predicted membrane localization, GM-NSC showed a PLPP4 nucleolar localization pattern consistent with that described in human cell lines (Human Protein Atlas, www.proteinatlas.org) and this pattern was maintained in sorted CD133⁺ cells (FIG. 4H). Another interesting candidate was FZDS, the putative receptor for Wnt5A which is involved in neural specification and highly expressed in the VZ (Bengoa-Vergniory et al., 2017. Mol. Neurobiol. 54, 6213-6224). GM-NSC showed a strong signal at the cell surface that was maintained in CD133⁺ GM-NSC (FIG. 4I). On the other hand, TREK-2 (KCNK10) a potassium channel that has been reported to be expressed by the ependymal cells (Prüss et al., 2011. Neuroscience 180, 19-29) and upregulated together with GFAP under ischemic conditions in astrocytes (Rivera-Pagán et al., 2015. PLoS One 10, 1-13) showed the expected membrane localization in GM-NSC but protein expression was attenuated following CD133 purification (FIG. 4J). Collectively these data indicate a distinctive GM-NSC signature independent of the contribution of more differentiated cell types in the starting samples.

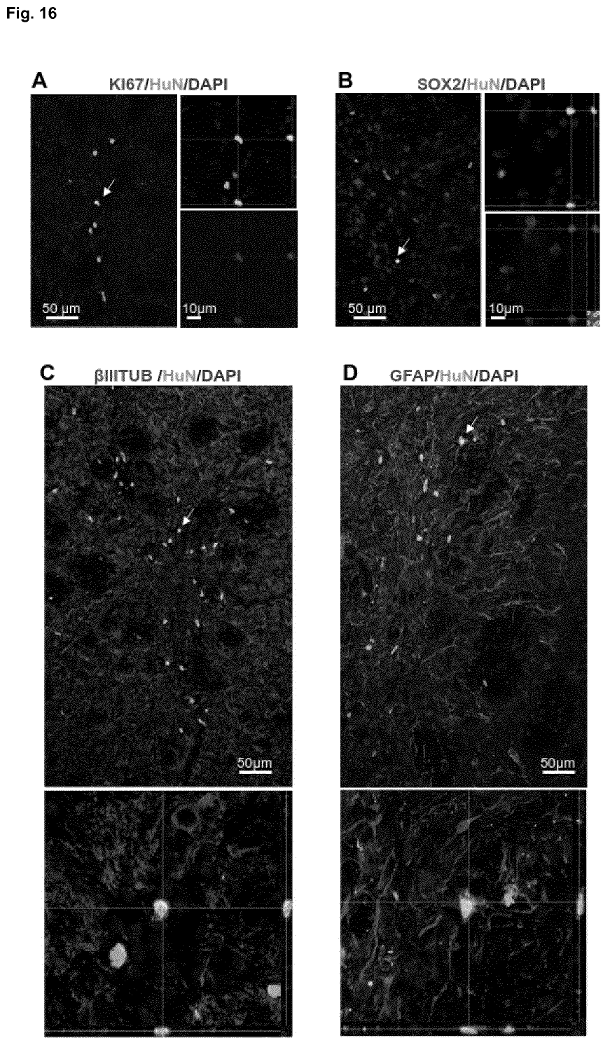

Finally, to evaluate the safety of GM-NSC, nude mice were transplanted with CD133⁺ purified cells in the striatum. GM-NSC remained in the tissue at and around the site of the injection 3 weeks after transplantation, showing no signs of tumor development or uncontrolled proliferation (FIG. 16). Transplanted cells showed little mitotic activity measured by K167 expression. Within the grafts we found SOX2 positive cells and more differentiated phenotypes in the neuronal (β-III-tubulin) and astrocytic (GFAP) lineages. None of the transplanted animals presented weight loss, neurological focal signs or any adverse reactions.

### Tables

TABLE 1

| Primary antibodies | Dilution | Company (Cat) |
|---|---|---|
| SOX2 | 1:500 | Chemicon (ab5603) |
| SOX1 | 1:100 | R&D system (af3369) |
| Nestin | 1:1000 | Abcam (ab22035) |
| β-III Tubulin | 1:1000 | Biolegend (801202) |
| GFAP | 1:2000 | Millipore (ab5804) |
| OLIG2 | 1:500 | Millipore (MABN50) |
| CD13 | 1:200 | BD Bioscience (555393) |
| Collagen I | 1:500 | Abcam (ab34710) |
| Fibronectin | 1:500 | ThermoFisher (MS-165-P0) |
| Ki67 | 1:100 | Dako (M7240) |
| Vimentin | 1:1000 | Abcam (AB20346) |
| Frizzled-5 | 1:200 | Novus (NBP2-37451) |
| GPR50 | 1:400 | Cell signalling (14032) |
| K2P10.1 (KCNK10, TREK2) | 1:100 | Alomone labs (APC-055) |
| PPAPDC1A | 1:10 | ThermoFisher (PA5-60944) |
| NCAM | 1:100 | Santa Cruz (sc-106) |
| Phalloidin | 1:40 | Invitrogen (A22287) |
| Flow cytometry antibodies | | |
| CD133 | 1:11 | Miltenyi (130-098-046) |
| CD24 | 1:11 | Miltenyi (130-099-118) |
| CD34 | 1:20 | Biolegend (343516) |
| CD45 | 1:20 | Biolegend (368516) |
| Podocalyxin | 1:11 | R&D (FAB1658P) |
| IL1RAP | 1:11 | R&D (FAB676G) |
| MHC II | 1:11 | Miltenyi (130-104-870) |
| Secondary antibodies | | |
| Donkey Anti-Rabbit IgG (H + L) | 1:200 | Invitrogen (A21206) |
| (Alexa -Fluor 488, green) | | |
| Donkey Anti-Rabbit IgG (H + L) | 1:200 | Invitrogen (A11012) |
| (Alexa -Fluor 594, red) | | |
| Donkey Anti-Goat IgG (H + L) | 1:200 | Invitrogen (A11058) |
| (Alexa-Fluor 594, green) | | |
| Donkey Anti-Mouse IgG (H + L) | 1.200 | Invitrogen (A11005) |
| (Alexa-Fluor 594, green) | | |
| Donkey Anti-Mouse IgG (H + L) | 1:200 | Invitrogen (A21206) |
| (Alexa-Fluor 488, green) | | |

TABLE 2
Brain 86 genes 51.49% p = 0.014
Genes: LYPD1, PRPH, MCHR1, CADM3, SHTN1, ZMAT4, IQGAP2, DLK1, APOBEC3C, KCNK10, ATCAY,
USP53, PAK3, IL1RAP, SERPINA3, SYNJ2, GNG3, GPC1, PTPRK, PARM1, SGK1, NRXN3, STMN2, KIF5A,
CECR1, CHODL, PCDH8, VAX1, ARHGAP29, MMP15, SLIT1, ELL2, EML1, TAGLN, ADGRV1, CNTN2, HLA-DPA1,
GUCY1B3, GPR17, KCNH2, SNX10, HLA-DRA, PLPP4, FAM69B, ADCYAP1R1, ABTB2, ADAMTS16,
NPY2R, SPOCK1, FHDC1, RIC3, CDH8, GALNT10, DGKG, GRPR, AHNAK2, HLA-DPB1, RAP1GAP2, PLXND1,
NEFL, CDC42EP3, MYOF, MYO5B, NEFM, ELMOD1, EHD4, SPP1, FLRT3, EPB41, MYO1B, EFEMP1, ATP11A,
IGSF9B, SFMBT2, MARCH3, FBLN1, NPY, SFRP1, ST8SIA4, TENM3, SLC6A6, MYO16, HBEGF, ADGRL2, MYLK,
IGFBP5
Hippocampus 16 genes 9.58% p = 1.21E−5
Genes: BCAT1, ABR, KIF5A, WBSCR17, NPY2R, ITGA3, SPOCK1, KCNIP4, NPY, SFRP1, IL1RAP, SERPINA3,
GPR17, KCNH2, MYLK, ELMOD1
Liver 28 genes 16.76% p = 0.038
Genes: ME1, MCHR1, HLA-DRB1, IQGAP2, CD74, APOD, CTGF, DGKG, IL1RAP, SERPINA3, SPRED3, CFI,
PLXND1, THBS1, SPP1, MGAT4A, PTPRK, PODXL, CECR1, ELL2, PLXDC2, DPYD, ADGRL2, IGFBP3, PROS1,
MYLK, EMP1, HLA-DRA
Plasma 11 genes 6.58% p = 2.48E−4
Genes: APOD, PLXDC2, CNTN2, SERPINA3, CFI, HLA-DMB, THBS1, PLXND1, PROS1, IGFBP3, IGFBP5
Lymphocytes 2 genes 1.19% p = 0.058
Genes: HLA-DRB1, HLA-DPB1

TABLE 3
Brain 131 genes 51.57% p = 7.055E−5
Genes: MOK, ADCY2, SNCAIP, SLC6A1, FGF11, PDE3B, SELENBP1, ITPKB, RORB, ZIC3, BBOX1, MAP3K5,
TIAM1, UNC5D, ROBO2, LGI1, ADGRB2, SPON1, MAP2K5, EGFR, SUCLG2, PLD5, ACTN2, NAV3, SSPN,
NCAM2, FAM222A, RGCC, ROR1, SLC25A37, EFNA5, UNC13A, ADAMTS4, PRODH, ERMP1, ADORA2B,
ERBB4, ARHGEF25, KCNA2, FAM212B, ASTN2, NRN1, RIMS1, ACSBG1, CSMD1, WDR54, HNMT, NDRG2,
ENTPD1, SGIP1, ZBTB47, OSBPL3, LAMTOR4, GSX2, CPNE5, TBC1D10A, SLC6A11, ABHD14A, AK4, ACACB,
DOCK5, TRIL, KCNK2, CAMK2N2, FEZF2, FAM198B, FAM214A, SYT14, KCTD15, QPRT, THNSL1, TMOD1,
KCNJ16, HIP1R, DPP10, PREX2, PAX6, NAP1L5, STXBP5L, KCNQ3, SLC24A3, SEMA3E, MGLL, CABLES1,
NRXN2, ALDH5A1, MPP2, PIK3C2B, OTX1, LDB2, NRXN1, GNAL, FOLH1, EYA2, CHRM3, CHST7, LIMCH1,
KCNH7, SUSD5, ANKFN1, ADAM19, ARL4D, ZNF483, SEZ6, GPAM, STON2, CLUAP1, CA14, ATP6V1G2,
PCDHB10, SAMD9L, PLPP2, AMPH, PGBD5, COL9A1, TSC22D3, MARVELD1, WDR17, FAT3, HSPA2, SNRK,
FAT4, COL27A1, ABCD2, SCG3, SNAP25, TBX2, ELAVL2, DBP, SNX32, SP8
Fetal brain 20 genes 7.87% p = 0.0039
Genes: MOK, SRPK2, NOL4, SNCAIP, MASP1, NRXN2, ERBB4, SLC6A11, LDB2, RBBP9, SGK223, TSC22D3,
RGS20, KCNQ3, TIAM1, RGCC, LGI1, SNAP25, MAP2K5, ADGRB2
Hippocampus 13 genes 5.11% p = 0.012
Genes: SLC16A3, ERMP1, GNAL, SUCLG2, DPP10, ADRA1A, TLR4, ITPKB, ASTN2, NDRG2, SNAP25, KCNK2,
SGIP1
Cerebellum 16 genes 6.29% p = 0.025
Genes: SNCAIP, ARHGEF25, PAX6, ITPKB, RORB, FAM212B, TLR4, NAP1L5, C1ORF226, ACSBG1, AMPH,
SLC16A3, RAB29, THNSL1, SEZ6, PRODH
Retina 10 genes 3.93% p = 0.03209079859171182
Genes: ISLR, RGS20, WDR17, LIMCH1, FGF11, SGCD, RORB, RORA, ARL4D, ZNF385A

TABLE 4
1. Higher in CSF- NSC (2 fold, FDR p < 0.05) 514
Astrocyte 193 genes 37.54% p = 3.60E−54
SPARC; TMEM200A; SERPINE1; SPATA20; SLC4A3; MYLK; DPYSL3; KDR; EPHB2; WLS; PKNOX2; EPHB3;
IER3; RBFOX2; P3H2; EML1; TM4SF1; PLPP4; RNF128; COL8A1; HRASLS; MYL9; CFI; LYPD1; THY1;
ADAMTS16; ADAMTS15; ADAMTS14; CHST15; INPP5J; NKX2-1; SYNDIG1; WWTR1; CADM3; CADM4;
GADD45A; LIF; NR2F1; RASSF8; GNG12; HSPA12A; ABTB2; FOSL1; ID3; IGSF9B; SLC44A5; DIRAS3;
PYGB; PTPRU; COL18A1; SERPINA3; TENM4; PTPRK; SHB; TIMP3; NEFL; TEAD3; GPR39; ANXA2;
TNFRSF12A; MAMDC2; MYOF; EMP1; GFRA1; SORCS3; TMEFF2; MMP19; CDC42EP1; MAPRE3;
HBEGF; PRICKLE2; FIGN; FAM129B; SRPX2; GPC1; FAM196A; PDPN; SPP1; FLNA; ANKRD52; CDKN2B;
PCGF2; SUSD1; VAX1; SULF2; DCBLD2; PDP1; DPY19L1; TMEM158; CLCF1; AGRN; ADGRL2; ADGRL3;
STEAP3; CSF1; IRS1; TUSC3; PROS1; NPY2R; CLEC18B; PLAT; OLFML2A; PVR; CLEC18A; GALNT10;
CTGF; SYDE1; EFEMP1; CCND1; PLAU; CAPN5; CAPN2; PHLDA2; STK32A; LZTS1; PHLDA1; POSTN;
G6PD; IGFBP5; IGFBP3; APLP1; MID1; SPOCD1; FRMD5; ADAM12; SCG2; RAI14; CHODL; FBN2; CUL7;
KHDRBS3; GLIS3; FBLN1; MCHR1; BAG3; NACC2; KIAA1644; NPTX2; PLXNA4; KLHL29; OSBPL5; BDNF;
TMEM132A; TNFRSF10C; ARHGAP29; GFAP; TNFRSF10D; BCL9; ULBP3; USP35; ITGB1; LGALS3BP;
ITGB4; CNRIP1; FHL2; FHL3; CYR61; SPRED3; ME1; PMEPA1; ITGA3; TMEM255A; PLEKHG5; TBC1D9;
ARAP3; C1QL4; C1QL1; PALLD; COL6A2; ITGA5; LAMA2; SRC; THBS1; NTN1; THBS3; MUC1; ATOH8;
NHS; SH3BP4; CCL2; SPOCK1; SLIT2; MEST; OPCML; SLC12A4; AHNAK2; LCA5; LAMB1; BAIAP2; DLK1;
MYO1B; MDGA1; BCAR1
Fibroblast 168 genes 32.68% p = 6.42E−38
STEAP3; SPARC; CSF1; IRS1; TUSC3; TMEM200A; PROS1; SERPINE1; PLAT; SPATA20; SLC4A3; PVR;
GALNT10; CTGF; ELK3; MYLK; SYDE1; EFEMP1; CCND1; PLAU; SCN9A; CAPN5; DPYSL3; CAPN2;
CCDC92; PHLDA2; PHLDA1; TNS3; WLS; IL13RA1; EPHB3; IER3; NOMO2; POSTN; G6PD; RBFOX2;
IGFBP5; IGFBP3; FNDC3B; P3H2; ACTN4; MID1; EML1; TM4SF1; SPOCD1; PLPP4; KCTD10; ADAM12;
EOGT; COL8A1; MYL9; RAI14; FBN2; CUL7; SDC4; USP53; GLIS3; ARHGAP18; FBLN1; THY1; SOCS3;
ADAMTS15; ADAMTS14; BAG3; NACC2; KIAA1644; S100A11; S100A10; KLHL29; WWTR1; OSBPL5;
BDNF; GADD45A; TMEM132A; LIF; NR2F1; ARHGAP29; RASSF8; SYNJ2; GNG12; L1CAM; HSPA12A;
ELL2; TRERF1; FOSL2; TNFRSF10D; FOSL1; ID3; ULBP3; USP35; ITGB1; PTPRU; COL18A1; HIBADH;
CNRIP1; FHL2; FHL3; ECE1; PTPRK; GRIK2; SHB; CYR61; SPRED3; FYCO1; LBH; TMEM43; ME1; TIMP3;
KIF1C; TEAD3; GPR39; ANXA2; TNFRSF12A; ITGA3; MAMDC2; MYOF; PLAUR; EMP1; GFRA1;
SLC39A14; TGFBR2; PALLD; OAF; COL6A2; CDC42EP3; MMP19; CDC42EP1; ITGA5; VAMP5; CD44;
SLFN5; LAMA2; PRICKLE2; THBS1; FAM129B; THBS3; SRPX2; GPC1; FAM196A; ATOH8; NHS; SH3BP4;
FLNA; CCL2; SPOCK1; CD59; SLIT2; ANKRD52; NQO1; CDKN2B; SLC12A4; PCGF2; AHNAK2; ATP2B4;
LCA5; LAMB1; DCBLD2; MYO1B; DPY19L1; TMEM158; CLCF1; PLP2; SEC24D; AGRN; PLCD3; MDGA1;
BCAR1; ADGRL2
Oligodendrocyte 158 genes 30.74% p = 3.51E32
RAB3C; SPARC; IRS1; PROS1; SERPINE1; NPY2R; CLEC18B; PLAT; SLC4A3; OLFML2A; HS6ST1; CTGF;
C4B; SYDE1; C4A; CCND1; CAPN5; DPYSL3; EPHB2; STK32A; PHLDA1; WLS; PKNOX2; EPHB3; KCNH2;
LMO1; ENTPD2; IGFBP5; IGFBP3; APLP1; P3H2; EML1; TM4SF1; FRMD5; PLPP4; EEF1A2; ADAM12;
SMS; HRASLS; MYL9; GRIA1; CHODL; FBN2; ADCYAP1R1; KHDRBS3; LYPD1; PDGFB; GLIS3; LPL; FBLN1;
THY1; MCHR1; ADAMTS16; ADAMTS15; ADAMTS14; FLRT3; PODXL; NACC2; KIAA1644; NKX2-1;
SYNDIG1; OTX2; NPTX2; B4GALNT3; LONRF2; PLXNA4; KLHL29; GPR17; WWTR1; CADM3; CADM4;
OSBPL5; SIAH3; TMEM132A; LIF; NR2F1; ARHGAP29; GNG12; L1CAM; HSPA12A; GFAP; DAB1;
COL20A1; IGSF9B; FGFR3; ULBP3; SLC44A5; DIRAS3; PYGB; PTPRU; COL18A1; ITGB4; PTPRO; CELF5;
GRIK2; SHB; CYR61; EPB41L4B; ME1; NEFL; TEAD3; CHST8; GPR39; MMP7; ADGRV1; TNFRSF12A;
ITGA3; TMEM255A; ELMOD1; MAMDC2; MYOF; EMP1; GFRA1; C1QL4; SORCS3; C1QL1; TMEFF2;
PALLD; COL6A2; MAPRE3; CRB2; PRICKLE2; FIGN; LRP2; NTN1; FAM129B; THBS3; PTCHD1; GPC1;
PDPN; SUSD4; SPP1; NHS; SLIT1; SH3BP4; FLNA; CCL2; SPOCK1; SLIT2; ANKRD52; PAK3; MEST;
OPCML; PCGF2; KCNIP4; AHNAK2; LCA5; BAIAP2; VAX1; PLEKHA7; SULF2; DCBLD2; PDP1; TMEM158;
ACKR1; AGRN; BCAR1; ADGRL3
Podocyte 150 genes 29.18% p = 7.07E−29
STEAP3; SPARC; CSF1; IRS1; TUSC3; SERPINE1; PLAT; SPATA20; SLC4A3; OLFML2A; PVR; GALNT10;
CTGF; MYLK; SYDE1; EFEMP1; CCND1; PLAU; DPYSL3; EPHB2; PHLDA2; LZTS1; IER3; POSTN; IGFBP5;
IGFBP3; P3H2; ACTN4; MID1; TM4SF1; RUNX2; SPOCD1; FRMD5; PLPP4; RNF128; EEF1A2; ADAM12;
COL8A1; HRASLS; MYL9; RAI14; FBN2; CUL7; SDC4; LYPD1; PDGFB; GLIS3; MCHR1; ADAMTS15;
SBNO2; ADAMTS14; NUAK2; NACC2; INPP5J; KLHL29; GPR17; WWTR1; CADM4; BTN2A2; BDNF;
GADD45A; TMEM132A; LIF; TNFRSF10C; ARHGAP29; TNFRSF10A; GNG12; L1CAM; HSPA12A; FOSL2
TNFRSF10D; ABTB2; FOSL1; DLG1; EHD4; MYO5B; BCL2L1; ULBP3; USP35; ITGB1; LGALS3BP; DIRAS3;
PTPRU; COL18A1; TENM4; ITGB4; CTSZ; FHL2; ECE1; PTPRK; SHB; CYR61; ME1; PMEPA1; NEFL;
TEAD3; GPR39; MMP7; TNFRSF12A; ITGA3; TMEM255A; PLEKHG5; ANXA4; MAMDC2; MYOF; GFRA1;
C1QL4; SORCS3; C1QL3; SLC39A14; C1QL1; CDCP1; PALLD; COL6A2; CDC42EP1; ARHGEF5; HBEGF;
SRC; PRICKLE2; FIGN; THBS1; NTN1; FAM129B; THBS3; GPC1; FAM196A; SUSD4; SPP1; NHS; SH3BP4;
FLNA; CCL2; SPOCK1; ANKRD52; TM4SF18; CDKN2B; SLC12A4; SMOX; PCGF2; SUSD1; AHNAK2; LCA5;
LAMB1; SULF2; DCBLD2; MYO1B; CLCF1; AGRN; MDGA1; BCAR1
2. Higher in fetal NSC (2 fold, FDR p < 0.05) 547
Fetal Brain 162 genes 29.62% p = 1.43E−29
SPON1; PID1; CPNE5; TRIL; TMEM200C; FAM107A; AQP4; RORB; RIMS1; SCGN; SYNPR; RIMS4; SOX1;
THSD7A; CPNE2; RGS7; UNC13A; DSCAM; KCNH7; CASK; ANK2; MMD2; SLC6A11; SRCIN1; LEMD1;
ENKUR; FOLH1; ADGRB2; DOK5; GPD1; ANOS1; AMPH; ARL8A; KCTD15; DLX2; NRN1; ARX; SLC1A2;
CDCA7; B3GLCT; FBLN2; RAP1GAP; ENHO; FAM171A1; SNN; NKAIN3; CDH20; FUT9; NDP; OTX1;
RAB6B; GRIA3; TBC1D16; ASIC1; SLC10A4; GLYATL2; GPR12; BTBD17; CADM2; AMBN; SLC4A10; MT3;
HOPX; ARHGAP31; KLHDC8A; CSPG5; SPIRE1; SP8; FAT4; SP9; EFCC1; SPRN; PTPRT; ROBO2; SNAP25;
DHRS13; ABCD2; COLGALT2; DOCK3; FAM69C; FAM57A; SLC6A1; SOBP; RND3; ELAVL2; RLBP1; GLI3;
CALB2; ZIC2; ZIC3; GRM8; PSD3; CNGA3; CA8; GAL3ST4; PRKG1; SNTA1; TIGD4; GPR37; CHST7;
ACTL6B; HMGCS1; ELOVL2; UBE2QL1; PAX6; TEX15; SEZ6L; IL17RD; EPN2; ARL4D; DCT; MPPED2;
FAM181B; GAS1; MAPT; RGS7BP; LRRC3B; PDZRN3; DSCAML1; ANGPTL1; PDZRN4; CRB1; HEPN1;
AMER2; SLC24A3; ZBTB47; LAMA1; NRXN1; KCNA2; DBX2; SEZ6; ATP1A2; LRP3; ABHD17C; ADCY8;
EGFR; DNAJB2; NWD2; ERBB4; CAMK2N2; SLITRK3; CLVS2; CSMD1; WASF3; MPP2; EYA2; KCNIP2;
B3GAT2; GAD1; GSX2; BBOX1; KLHL4; ZIC5; SNCAIP; STXBP5L; APC; PSAT1; HCG22; RGS11; ZNF853;
KCNK2; CDK5R1
Cerebral Cortex 152 genes 27.79% p = 1.08E−24
PID1; CPNE5; DGKB; TRIL; MT1M; TMEM200C; FAM107A; ARHGDIG; AQP4; RORB; ENO2; PREX2;
RIMS1; SYNPR; SOX1; THSD7A; PTGDS; PDE8B; STK32C; RGS7; PAQR8; UNC13A; DSCAM; KCNH7;
TMOD2; DIO2; ANK2; MMD2; SLC6A11; GPR37L1; SRCIN1; MTURN; ENKUR; CLDN5; RAB30; FOLH1;
ADGRB2; DOK5; RUFY3; SCG3; AMPH; ZFPM2; NRN1; HEPACAM; ARX; SLC1A2; CWF19L2; NDRG2;
RAP1GAP; ENHO; FAM171A1; SNN; NKAIN3; CDH20; FUT9; NDP; OTX1; RAB6B; GRIA3; ASIC1; GPR12;
BTBD17; HOMER1; CADM2; SLC4A10; MT3; COPRS; CPEB1; FXYD1; CSPG5; SPIRE1; BMPR1B;
OSBPL1A; SPRN; PTPRT; ROBO2; SNAP25; ABCD2; COLGALT2; DOCK3; FAM69C; BHLHE41; SLC6A1;
SOBP; ELAVL2; RLBP1; CALB2; ZIC2; GRM8; PSD3; CA8; GPRASP1; PRKG1; SNTA1; GPR37; ACTL6B;
SGIP1; ELOVL2; UBE2QL1; PAX6; TEX15; SEZ6L; GNL1; IL17RB; F8; DCT; FAM181B; ACSBG1; MAPT;
RGS7BP; LRRC3B; DSCAML1; PDZRN4; CRB1; HEPN1; AMER2; SLC24A3; ZBTB47; NRXN1; KCNA2;
DBX2; SEZ6; AKAP7; ATP1A2; SNX32; EGFR; DNAJB2; NWD2; ERBB4; CAMK2N2; SLITRK3; CLVS2;
PTK2B; CSMD1; WASF3; SASH1; RFTN2; CGREF1; MPP2; EYA2; LGI4; KCNIP2; B3GAT2; GAD1; BBOX1;
KLHL4; STXBP5L; TEF; APC; ZNF853; KCNK2; CDK5R1
Prefrontal Cortex 148 genes 27.06% p = 6.50E23
PID1; CPNE5; DGKB; TRIL; RASEF; TMEM200C; FAM107A; ARHGDIG; AQP4; RORB; ENO2; PREX2;
RIMS1; SYNPR; RIMS4; RASSF2; SOX1; THSD7A; PTGDS; RGS7; PDK1; UNC13A; SLC15A2; DSCAM;
KCNH7; TMOD2; ACSL6; DIO2; CASK; ANK2; MMD2; SLC6A11; GPR37L1; SRCIN1; CACNB2; CLDN5;
ADGRB2; DOK5; KAT6B; RUFY3; KIAA1456; ZNF713; SCG3; MASP1; DLX2; HEPACAM; ARX; SLC1A2;
NREP; EFNA5; ENHO; FAM171A1; NKAIN3; CDH20; FUT9; TTPA; NDP; OTX1; RAB6B; GRIA3; ASIC1;
ZNF781; GPR12; CADM2; SLC4A10; MT3; HOPX; CPEB1; KLHDC8A; COL9A1; CSPG5; SPIRE1; SP8;
FAT4; BMPR1B; SP9; PTPRT; ROBO2; SNAP25; DOCK3; FAM69C; DOCK7; SLC6A1; SOBP; RND3;
ELAVL2; RLBP1; CALB2; NADK2; SEPT7; ZIC2; PSD3; ZNF404; ACTL6B; HMGCS1; SGIP1; ELOVL2;
UBE2QL1; PAX6; SEZ6L; IL17RD; EPN2; RILPL1; ZADH2; MPPED2; FAM181B; ACSBG1; MAPT; RGS7BP;
LRRC3B; PDZRN3; DSCAML1; ASPA; PDZRN4; CRB1; HEPN1; AMER2; NRXN1; KCNA2; DBX2; SEZ6;
ATP1A2; ADCY8; FGD4; NWD2; ERBB4; CAMK2N2; SLITRK3; CLVS2; APBB2; CSMD1; RFTN2; NEGR1;
LGI4; B3GAT2; GAD1; BBOX1; SYT14; KLHL4; SNCAIP; KIAA1161; FAM213A; STXBP5L; MGAT4C;
KBTBD6; APC; KCNK2; CDK5R1
Spinal Cord 133 genes 24.31% p = 1.72E−16
SPON1; DGKB; TRIL; TMEM200C; AQP4; ENO2; PREX2; RIMS1; SYNPR; RIMS4; SOX1; FAM198B;
THSD7A; PDE8B; RGS7; UNC13A; DSCAM; UNC5B; TMOD2; ANK2; MMD2; SLC6A11; SRCIN1; MTURN;
ENKUR; CLDN5; FOLH1; ADGRB2; DOK5; ANOS1; AMPH; MASP1; KCTD15; NRN1; SLC1A2; FBLN2;
RAP1GAP; ENHO; FAM171A1; SNN; NKAIN3; CDH20; FUT9; NDP; RAB6B; GRIA3; TBC1D16; ASIC1;
SLC10A4; BTBD17; CADM2; SLC4A10; MT3; KLHDC8A; FXYD1; PDE3A; CSPG5; SP8; FAT4; SP9; SPRN;
PTPRT; ROBO2; SNAP25; COLGALT2; DOCK3; FAM69C; SLC6A1; SOBP; ELAVL2; RLBP1; GLI3; CALB2;
ZIC2; ZIC3; GRM8; CNGA3; GPRASP1; PRKG1; TIGD4; GPR37; ACTL6B; SGIP1; ELOVL2; UBE2QL1;
SEZ6L; IL17RD; MPPED2; FAM181B; GAS1; MAPT; RGS7BP; LRRC3B; PDZRN3; DSCAML1; PDZRN4;
AMER2; SLC24A3; ZBTB47; LAMA1; NRXN1; KCNA2; DBX2; ATP1A2; LRP3; ADCY8; EGFR; DNAJB2;
NWD2; ERBB4; CAMK2N2; SLITRK3; CLVS2; CSMD1; WASF3; SASH1; MPP2; EYA2; LGI4; KCNIP2;
B3GAT2; GAD1; GSX2; BBOX1; KLHL4; ZIC5; SNCAIP; STXBP5L; HCG22; RGS11; ZNF853; KCNK2;
CDK5R1

TABLE 5

| | Dilution | Company (Cat) |
|---|---|---|
| Primary antibodies | | |
| SOX2 | 1:500 | Chemicon (ab5603) |
| Nestin | 1:1000 | Abcam (ab22035) |
| BLBP | 1:400 | R&D (ABN14) |
| β-III Tubulin | 1:1000 | Biolegend (801202) |
| GFAP | 1:2000 | Millipore (ab5804) |
| OLIG2 | 1:500 | Millipore (MABN50) |
| CD13 | 1:200 | BD Bioscience (555393) |
| Collagen I | 1:500 | Abcam (ab34710) |
| Fibronectin | 1:500 | ThermoFisher (MS-165-P0) |
| Ki67 | 1:100 | Dako (M7240) |
| Vimentin | 1:1000 | Abcam (AB20346) |
| Frizzled-5 | 1:200 | Novus (NBP2-37451) |
| TREK2 | 1:100 | Alomone labs (APC-055) |
| PPLP4 | 1:10 | ThermoFisher (PA5-60944) |
| HuNu | 1:300 | Millipore |
| Flow cytometry antibodies | | |
| CD133-PE | 1:11 | Miltenyi (130-098-046) |
| CD24-FITC | 1:11 | Miltenyi (130-099-118) |
| CD34-PECy7 | 1:20 | Biolegend (343516) |
| CD45-APCCy7 | 1:20 | Biolegend (368516) |
| Podocalixyn-PE | 1:11 | R&D (FAB1658P) |
| IL1RAP-AF488 | 1:11 | R&D (FAB676G) |
| MHC-II-APC | 1:11 | Miltenyi (130-104-870) |
| Secondary antibodies | | |
| Donkey Anti-Rabbit IgG (H + L) (Alexa - | 1:200 | Invitrogen (A21206) |
| Fluor 488, green) | | |
| Donkey Anti-Rabbit IgG (H + L) (Alexa - | 1:200 | Invitrogen (A11012) |
| Fluor 594, red) | | |
| Donkey Anti-Goat IgG (H + L) (Alexa-Fluor | 1:200 | Invitrogen (A11058) |
| 594, green) | | |
| Donkey Anti-Mouse IgG (H + L) (Alexa- | 1.200 | Invitrogen (A11005) |
| Fluor 594, green) | | |
| Donkey Anti-Mouse IgG (H + L) (Alexa- | 1:200 | Invitrogen (A21206) |
| Fluor 488, green) | | |

TABLE 6

| Gene | Primer Sequence |
|---|---|
| CD133 | F: 5′-CACCGCTCTAGATACTGCTGTTGA-3′ |
| | R: 5′-TGATGGACCATGGACTATAACGTG-3′ |
| SOX2 | F: 5′-AGAAGAGGAGAGAGAAAGAAAGGGAGAGA-3′ |
| | R: 5′-GAGAGAGGCAAACTGGAATCAGGATCAAA-3′ |
| FABP7 | F: 5′-AAGGATGGTGGAGGCTTTCT-3′ |
| | R: 5′-TTTGGTCACATTTCCCACCT-3′ |
| DCX | F: 5′-CATCCCCAACACCTCAGAAG-3′ |
| | R: 5′-GGAGGTTCCGTTTGCTGA-3′ |
| MAP2 | F: 5′-CTAACCGAGGAAGCATTG-3′ |
| | R: 5′-TTCTCCTGCAACTATTCAAG-3′ |
| SOX1 | F: 5′-ATTATTTTGCCCGTTTTCCC-3′ |
| | R: 5′-TCAAGGAAACACAATCGCTG-3′ |
| GFAP | F: 5′-TCTCTCGGAGTATCTGGGAACTG-3′ |
| | R: 5′-TTCCCTTTCCTGTCTGAGTCTCA-3′ |
| FOXG1 | F: 5′-TTCAGCTACAACGCGCTCAT-3′ |
| | R: 5′-ACAGATTGTGGCGGATGGAG-3′ |
| PODXL | F: 5′-CTCACCGGGGACTACAACC-3′ |
| | R: 5′-GCCTCCTCTAGCCACGGTA-3′ |
| PLPP4 | F: 5′-TTTGGATCCGTTCCAGAGAG-3′ |
| | R: 5′-CAGGGGTGTGAGGAAAGAAA-3′ |
| KCNK10 | F: 5′-AAGCATGGGCAGGGTGCGTC-3′ |
| | R: 5′-TCCGGCTCCCGGTCTTTGGT-3′ |
| FZD5 | F: 5-TGTCTGCTCTTCTCGGC-3′ |
| | R: 5′-CCGTCCAAAGATAAACTGCT-3′ |
| IL1RAP | F: 5′-GGGACTAGACACCATGAGGCAAAT-3′ |
| | R: 5′-TGCCTAGTCCAATACCAGATCAGAG-3′ |
| HLA-DRA | F: 5′-GCTATCAAAGAAGAACATGTG-3′ |
| | R: 5′-GAGCGCTTTGTCATATTTCCAG-3′ |
| HLA-DQA1 | F: 5′-GAGCAGTTCTACGTGGACCTGG-3′ |
| | R: 5′-GGAACCTCATTGGTAGCAGCA-3′ |
| HLA-DPA1 | F: 5′-TGGCTGACTGAATTGCTGAC-3′; |
| | R: 5′TGAGGGGTTCTTCAAAGGAG-3′ |
| ACTIN | F: 5′-TGAAGTGTGACGTGGACATC-3′ |
| | R: 5′-GGAGGAGCAATGATCTTGAT-3′ |

### CLAUSES

1. A purified or enriched cell population which comprises at least 40% stem cells obtained from the cerebrospinal fluid (CSF) of premature babies with intraventricular haemorrhage, wherein said stem cells are characterized by being positive to CD133, and optionally CD34 and CD24 and negative for CD45.
2. The population of claim 1, wherein said stem cells are obtained from the ventricle cavity of premature babies, preferably from the ventricle cavity with the larger amount of hematoma, wherein said ventricle is preferably punctured with the surgical endoscope under intraoperative ultrasound guidance.
3. The population of any of claim 1 or 2, wherein said stem cells are further characterized by being positive by inmunofluorescence to the expression of one or more of the following markers Sox1, Sox2, Ki67, Nestin, and vimentin markers; and negative for fibroblast markers CD13, Collagen I and Fibronectin.
4. The population of any of claims 1 to 3, wherein said stem cells are further characterized by overexpressing Podocalyxin, KCNK10, PLPP4, GPR50, HLA-DR-A, HLA-DP-A1, HLA-DQ-A1 and IL1RAP in comparison to foetal NSCs; and wherein FGF11, TIAM1, EGFR, NCAM2, ADAMTS4 and ADAM19 are downregulated genes when compared with foetal NSCs.
5. A purified or enriched cell population which comprises at least 40% stem cells obtained from the cerebrospinal fluid (CSF) of premature babies with intraventricular haemorrhage, wherein said stem cells are characterized by:
   a. being positive to CD133, and optionally CD34 and CD24 and negative for CD45;
   b. being positive by inmunofluorescence to the expression of Sox1, Sox2, Ki67, Nestin and vimentin markers, and negative for the fibroblast markers CD13, Collagen I and Fibronectin; and
   c. by overexpressing Podocalyxin, KCNK10, PLPP4, GPR50, HLA-DR-A, HLA-DP-A1, HLA-DQ-A1 and IL1RAP in comparison to foetal NSCs; and
   d. wherein FGF11, TIAM1, EGFR, NCAM2, ADAMTS4 and ADAM19 are downregulated genes when compared with foetal NSC.
6. A composition adapted for and suitable for delivery to a patient, i.e., physiologically compatible which comprises the purified or enriched cell population of any of claims 1 to 5.
7. The composition according to claim 6, wherein said composition is a pharmaceutical composition which optionally comprises a carrier and/or pharmaceutically acceptable excipients.
8. The composition of any of claim 6 or 7, wherein said composition comprises one or more of buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.
9. The composition of any of claims 6 to 8, wherein said composition is adapted for or suitable for freezing or storage.
10. The composition of any of claims 6 to 9, for use in methods of treating or preventing injuries and diseases or other conditions.
11. The composition for use according to claim 10, wherein the cell population of the composition is obtained using a tissue sample obtained from the patient being treated (autologous treatment).
12. The composition for use according to claim 10, wherein the cell population of the composition is obtained from a donor, who may be related or unrelated to the patient (i.e., allogeneic treatment), and wherein the donor is of the same species as the patient or of a different species (i.e., xenogeneic treatment).
13. The composition for use according to any of claims 10 to 12, for use in the treatment or prevention of inflammatory diseases, demyelinating diseases, mental disorders, neurodegenerative diseases such as ELA, Alzheimer or Parkinson, neuromuscular diseases.
14. The composition for use according to any of claims 10 to 12, for use in the treatment or prevention of premature babies having or suffering from Intraventricular haemorrhage or post-hemorrhage hydrocephalus.
15. The composition for use according to claim 14, wherein the treatment is an autologous treatment.

1. A cell population which comprises at least 40% stem cells obtained from the cerebrospinal fluid (CSF) of premature babies with intraventricular haemorrhage, wherein said stem cells are characterized by being positive to CD133, and optionally CD34 and CD24 and negative for CD45.

2. The population of claim 1, wherein said stem cells are obtained from the ventricle cavity of premature babies, wherein said ventricle is preferably punctured with the surgical endoscope under intraoperative ultrasound guidance.

3. The population of any of claim 1 or 2, wherein said stem cells are further characterized by being positive by inmunofluorescence to the expression of one or more of the following markers, Sox2, Ki67, Nestin, and vimentin markers; and negative for fibroblast markers CD13, Collagen I and Fibronectin.

4. The population of any of claims 1 to 3, wherein said stem cells are further characterized by overexpressing PODXL, IL1RAP, HLA-DR, and FZDS.

5. The population of any of claims 1 to 4, wherein said stem cells are further characterized by overexpressing Podocalyxin, KCNK10, PLPP4, GPR50, HLA-DR-A, HLA-DP-A1, and IL1RAP in comparison to foetal NSCs.

6. The population according to claim 5, wherein said stem cells are further characterized by overexpressing HLA-DQA1.

7. The population of any of claims 1 to 6, wherein ZIC3, TIAM1, EGFR, PAX6, AQP4 or GSX2 are downregulated genes when compared with foetal NSCs.

8. A cell population which comprises at least 40% stem cells obtained from the cerebrospinal fluid (CSF) of premature babies with intraventricular haemorrhage, wherein said stem cells are characterized by:
a. being positive to CD133, and optionally CD34 and CD24 and negative for CD45;
b. being positive by inmunofluorescence to the expression of Sox1, Sox2, Ki67, Nestin and vimentin markers, and negative for the fibroblast markers CD13, Collagen I and Fibronectin; and
c. by overexpressing Podocalyxin, KCNK10, PLPP4, GPR50, HLA-DR-A, HLA-DP-A1, HLA-DQ-A1 and IL1RAP in comparison to foetal NSCs; and
d. wherein ZIC3, TIAM1, EGFR, AQP4, PAX6, or GSX2 are downregulated genes when compared with foetal NSC.

9. The cell population according to claim 8, wherein the stem cells are characterized also by overexpressing FZDS.

10. A composition adapted for and suitable for delivery to a patient, i.e., physiologically compatible, which comprises the purified or enriched cell population of any of claims 1 to 9.

11. The composition according to claim 10, wherein said composition is a pharmaceutical composition which optionally comprises a carrier and/or pharmaceutically acceptable excipients.

12. The composition of any of claim 10 or 11, wherein said composition comprises one or more of buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

13. The composition of any of claims 10 to 12, wherein said composition is adapted for or suitable for freezing or storage.

14. The composition of any of claims 10 to 13, for use in methods of treating or preventing injuries and diseases or other conditions.

15. The composition for use according to claim 14, wherein the cell population of the composition is obtained using a tissue sample obtained from the patient being treated (autologous treatment).

16. The composition for use according to claim 14, wherein the cell population of the composition is obtained from a donor, who may be related or unrelated to the patient (i.e., allogeneic treatment), and wherein the donor is of the same species as the patient or of a different species (i.e., xenogeneic treatment).

17. The composition for use according to any of claims 14 to 16, for use in the treatment or prevention of inflammatory diseases, demyelinating diseases, mental disorders, neurodegenerative diseases such as ELA, Alzheimer or Parkinson, neuromuscular diseases.

18. The composition for use according to any of claims 14 to 16, for use in the treatment or prevention of premature babies having or suffering from Intraventricular haemorrhage or post-hemorrhage hydrocephalus.

19. The composition for use according to claim 18, wherein the treatment is an autologous treatment.
